(19) United States
(12) Patent Application Publication
(10) Pub. No.: US 2011/0245662 A1
(43) Pub. Date: Oct. 6, 2011

(54) Hemodynamic Detection of Circulatory Anomalies

(76) Inventors: Philip E. Eggers, Dublin, OH (US); Andrew R. Eggers, Ostrander, OH (US); Eric A. Eggers, Portland, OR (US); Michael W. Jopling, Columbus, OH (US)

(21) Appl. No.: 12/754,888

(22) Filed: Apr. 6, 2010

(51) Int. Cl. A61B 5/0275; A61B 6/00

(52) U.S. Cl. 600/431; 600/508

(57) ABSTRACT

The invention generally relates to a system, method and apparatus for detection of circulatory anomalies in the mammalian body. Particularly, apparatus is provided that allows the clinician to quantitatively determine the extent of any anomalies in the pulmonary circulation. Specifically a quantifiable agent is injected into a peripheral location, and the transit of the indicator agent is monitored. Aberrant circulation is them quantified. The preferred indicator is an injection of indocyanine green dye, detected and measured by fluorescence at a sensor location, for example, at the human ear. Quantification is carried out by using cardiac output procedures and alternatively, the use of Valsalva Maneuver is monitored at a monitor/controller providing visual cues to the patient and operator.

### CROSS-REFERENCE TO RELATED APPLICATIONS

Not applicable.

### STATEMENT REGARDING FEDERALLY SPONSORED RESEARCH

Not applicable.

### BACKGROUND

The present invention generally relates to a system, method and apparatus for detection of circulatory anomalies in the mammalian body. Important ones of such anomalies are generally referred to as cardiac right-to-left shunts.

An anomaly commonly encountered in humans is an opening between chambers of the heart, particularly an opening between the left and right atria, i.e. a right-left atrial shunt, or between the left and right ventricles, i.e. a right-left ventricular shunt. The shunt may occur as a defect within the vasculature leading to and from the heart, for example a Pulmonary Arteriovenous Malformation (PAVM) may be present as an open hole shunting between vein and artery. Over 780,000 patients suffer strokes each year in the U.S. resulting in 250,000 stroke related deaths. The total cost associated with stroke is reported to be $66 billion in the U.S. in 2007 (Rosamond 2008). Of the patient population presenting with stroke or the early warning sign known as transient ischemic attack (TIA or mini stroke), as many as 260,000 are reported to be the result of a right-to-left shunt in the heart and/or pulmonary vasculature.

The most common form of right-to-left shunt is the patent foramen ovale (PFO) which is an opening in the wall of the heart which separates the right side of the heart from the left side of the heart. The right side of the heart receives oxygen-depleted blood from the body and then pumps this blood into the lungs for reoxygenation. The lungs not only reoxygenate the blood, but also serve as a “filter” for any blood clots and also serves to metabolize other agents that naturally reside within the venous blood. During the fetal stage of development, an opening naturally exists between the right and left side of the heart to enable circulation of the mother's oxygen-rich blood throughout the vasculature of the fetus. This opening between the right and left side of the fetus' heart (known as the foramen ovale) permanently seals shut in consequence of the closure of a tissue flap in about 80% of the population within the first year following birth. Often the noted flap remains in a sealing orientation because of a higher pressure at the left side of the heart. However, in the remaining 20% of the population, this opening fails to permanently close which is referred to as a patent foramen ovale or PFO.

Most of the population exhibiting a PFO never experience any symptoms or complications associated with the presence of a PFO since many PFOs are small enough to remain effectively “closed.” However, for some subjects, this normally closed flap (i.e., foramen ovale) temporarily opens allowing blood to flow directly from the right side to the left side of the heart. As a consequence, any blood clots or other active agents escaping through the PFO bypass the critical filtering functions of the lungs and flow through the brief opening in this flap and directly to the left side of the heart. Once in the left side of the heart, any unfiltered blood clots or metabolically active agents pass directly into the arterial circulatory system. Since a significant portion of the blood exiting the left side of the heart flows to the brain, any unfiltered blood clots or agents such as serotonin may be delivered to the brain. Presence of these substances in the brain arterial flow can produce debilitating and life-threatening consequences. These consequences are known to include stroke, heart attack and are also now believed to be one of the causes of certain forms of severe migraine headaches. For further background on circulatory anomalies, see:

1) Banas, J., et al. American Journal of Cardiology 28: 467-471 (October 1971);

2) Castillo, C., et al. American Journal of Cardiology 17: 691-694 (May 1966);

3) Schwedt, T. J., et al., “Patent Foramen Ovale Migraine—Bringing Closure to the Subject.” Headache 46(4): 663-671 (2006).

4) Spies, C., et al., “Transcatheter Closure of Patent Foramen Ovale in Patients with Migraine Headache.” Journal of Interventional Cardiology 19(6): 552-557 (2006).

A relatively large number of patients (three million) have or may be undergoing sclerotherapy treating, for instance, varicose veins. This therapy involves an injection of sclerosing solution which in effect creates emboli. If patients undergoing sclerotherapy are among the proportion of the population with a PFO, creation of emboli that may bypass the filtering aspect of the lungs creates a significant risk of initiating a TIA, stroke or heart attack. This risk could be avoided by effectively and efficiently screening for a right-to-left shunt.

Based on the growing clinical evidence linking strokes, transient ischemic attacks (TIAs) and migraine headaches to right-to-left shunts, at least 16 companies have now entered the field of transvascular shunt treatment devices for closure of the most common form, viz., a patent foramen ovale (PFO), and certain of these devices are approved for sale in one or more principalities.

Percutaneous closure devices are expected to soon be widely available in the U.S. for PFO closure, and over 10% of the adult population is estimated to have a congenital patent foramen ovale (PFO). Unfortunately, there is currently no available method suitable for widespread screening for the presence of a PFO when the patient experiences early warning signs signaling an ischemic incident, or the patient exhibits or is exposed to an elevated risk of a stroke. Consequently, the “at risk” fraction of the population with a right-to-left shunt is most often resigned to the possibility of experiencing a stroke before definitive right-to-left shunt testing is performed. Only then are methods such as transesophageal echocardiography (TEE) performed to detect the possible presence of a right-to-left shunt. If detected, the patient may elect one of a growing number of transcatheter right-to-left shunt closure procedures or the more conventional open-heart procedure for right-to-left shunt closure.

Transesophageal echocardiography (TEE) is resorted to somewhat as a last resort. It is considered the “gold standard” of determining the presence of a right-to-left shunt. In carrying out this test, microbubbles are injected into a vein leading to the right side of the heart. As this is underway, the patient is required to blow into a manometer to at least a pressure of 40 mm of mercury (Valsalva Maneuver). Simultaneously, a sonic detector is held down the throat to record the passage of the microbubbles across the shunt. Because of gagging problems, the patient is partially anesthetized. Typically, patients will refuse to repeat the painful test and it is hardly suited for screening. The TEE test is expensive with an equipment total cost of between $75,000 and $322,000. It additionally requires a physician with a specialized two year fellowship and an anesthesiologist.

Another test is referred to as transthoracic echocardiography (TTE). Again, microbubbles are injected into a vein leading to the right side of the heart. The Valsalva Maneuver is carried out and ultrasonic echograms are made at the chest wall. The procedure requires the use of expensive equipment and exhibits about a 60% sensitivity.

A third test again uses microbubbles as a contrast agent along with the Valsalva Maneuver. Here, however, the ultrasonic sensors perform in conjunction with the temporal artery usually at both sides of the head. This transcranial doppler method (TCD) exhibits a high sensitivity and costs between about $30,000 to $40,000 for equipment. Unfortunately, over 20% of the population has a cranial bone that's too thick for sonic transducing. U.S. Patent Publication US2006/0264759 describes such systems and methods for grading microemboli in blood associated with ultrasound contrast agenda (e.g., small air bubbles) within targeted vessels by using Doppler Ultrasound system.

Additional description of existing methods of analyzing circulation and detecting certain circulatory anomalies are present in the following.

5) Swan, H. J. C., et al., “The Presence of Venoarterial Shunts in Patients with Interatrial Communications.” Circulation 10: 705-713 (November 1954);

6) Kaufman, L., et al., “Cardiac Output Determination by Fluorescence Excitation in the Dog.” Investigative Radiology 7: 365-368 (September-October 1972);

7) Karttunen, V., et al. Acta Neurologica Scandinavica 97: 231-236 (1998);

8) Karttunen, V., et al., “Ear Oximetry: A Noninvasive Method for Detection of Patent Foramen Ovale—A Study Comparing Dye Dilution Method and Oximetry with Contrast Transesophageal Echocardiography.” Stroke 32(2): 32: 445-453 (2001).

A continuing difficulty with existing methods is the efficacy of using microbubbles as a circulatory tracking indicator. Microbubbles are created just prior to use, are a transient structure, and decidedly non-uniform in creation and application. It is difficult if not impossible for microbubbles to be used for quantitative measurements, and thus clinicians are forced to rely on a positive or negative result assessment. In part, the inability to effectively quantify the conductance of a shunt is revealed in the relatively low sensitivity of the existing methods.

A further problem with existing methods is the difficulty in effectively detecting the circulatory tracking indicator in the form of microbubbles. Each of existing methods, including transesophageal echocardiography, transthoracic echocardiography, and the transcranial doppler method suffer from barriers for routine use for screening, whether due to the need for anesthesia or expensive equipment. There is a need for more efficient circulatory tracking reagents, i.e. a reagent that can be reproducibly introduced into the circulatory system, be quantitatively detectable, and utilize relatively straightforward detection systems that are easily tolerated by patients.

One difficulty with improving the present technology in circulatory tracking reagents is that there heretofore has been no animal model available for screening a variety of different circulatory tracking reagents and their compatible detection systems.

There exists a growing body of clinical evidence linking the presence of right-to-left shunts to the risk of embolic strokes and occurrence of migraine headaches. In spite of this evidence, there remains a significant unmet need for a high sensitivity, low-cost and non-invasive method to screen those patients at increased risk of stroke in order to detect PFOs or other circulatory anomalies. The ability to screen at-risk patients is a critically unmet need, since shunt-related strokes can only be prevented if the presence of the shunt is detected and closed in advance of the occurrence of a stroke. In addition, there is likewise a significant unmet need for a highly sensitive, quantitative low-cost method for evaluating the effectiveness and durability of the closure at 3 to 4 time points following the percutaneous closure of the right-to-left shunt. This follow-up testing following shunt closure continues to be essential for assuring adequacy of the “seal” closing a PFO or other shunt, in order to minimize the risk of future shunt-related strokes.

In application for U.S. patent Ser. No. 12/418,866, a generally non-invasive technique for screening for circulatory anomalies such as patent ovale foramen is disclosed. With the system and method, a fluorescing indicator (indocyanine green dye) is injected within the venous system and a resultant dilution curve is detected at the arterial vasculature in the pinna of the ear. In general, a red region laser beam is applied at the ear surface in a reflection operational mode and the indicator photons emitted in fluorescence are filtered and measured for intensity. This results in one or more intensity curves, an initial one being in response to a shunt condition and the subsequent curve representing a larger concentration resulting from passage of the indicator through the lungs and back through the heart. In this regard, if a shunt condition is present, the intensity read-out will generate a lower intensity preliminary shunt curve. This will be followed by the noted larger dilution curve.

With the encouragement of the somewhat extensive animal (pig) data, it now becomes necessary to improve fluorescing photon intensity measurement and to explore human physiology with respect to the transit of the indicator, its optimum injection site and timing, the use of the characteristics of a Valsalva Maneuver, improving fluorescing photon intensity measurement as well as overall testing reliability. This called for bench-testing for sensor optimization, extensive medical literature searching to improve the overall procedure and additional animal (pig) as well as human trials.

### BRIEF SUMMARY

The present system is addressed to system, method and apparatus for detecting and quantifying right-to-left pulmonary shunts. The preferred indicator which is employed is indocyanine green dye (ICG) which will fluoresce when exposed to an appropriate wavelength of higher energy light, for example, a laser in the red region. The procedure is under the control of a monitor/controller having a visual display and capable of providing cues to both the operator and the patient. A vein access catheter is employed in connection with a peripheral vein such as the antecubital vein in an arm. Sensing of the indicator concentration takes place at an arterial vasculature, preferably at the pinna of the human ear. The system performs using fluorescence sensor arrays each with three indicator fluorescing lasers, which are directed to an artery of the scaphoid fossa of the ear pinna, where relatively thin tissue contains an arterial blood network. These sensors are configured for transmission mode measurement wherein three lasers are combined with aspheric collimating lenses for positioning at one side of the ear and at the opposite side of the ear tissue, there is positioned a photon collimating orifice and an optical band pass filter, selected to permit only fluorescing photons to reach a photodetector. The two branches of these fluorescence sensor array configurations are preferably spring biased to be held improper and stable positions at the ear.

The preferred method preferably incorporates a Valsalva Maneuver of about six seconds duration, during which two protocols for controls over injection of indicator may be carried out for a given session. As an adjunct to the control system, a Doppler ultrasound arrangement is utilized with a pickup positioned on the left parosternal position of the chest. This provides an output signal corresponding with the movement of normal saline solution into the right side of the heart. To assure proper termination of the Valsalva Maneuver, a solenoid actuated pneumatic valve may be incorporated in the monitor/controller, to release pressure in an exhalation tube at the proper instant in the procedure.

The monitor/controller may be configured to calculate an area under a normal indicator/dilution curve associated with indicator and blood flow through a normal pathway in the lungs. Additionally, the monitor/controller can calculate the area under any premature indicator dilution curve, which will be associated with a right-to-left shunt. The monitor/controller further corrects the main indicated curve for a recirculation phenomenon and to quantify any right-to-left shunt, calculates conductance associated with such shunts.

Other objects of the invention will, in part, be obvious and will, in part, appear hereinafter. The various embodiments of the invention, accordingly, comprises the method, apparatus and system possessing the construction, combination of elements, arrangement of parts and steps which are exemplified in the following detailed description.

### BRIEF DESCRIPTION OF THE DRAWINGS

For a full understanding of the nature and objects of the various embodiments of the invention, reference should be had to the following detailed description taken in connection with the accompanying drawings, in which:

FIG. 1 is a schematic and sectional representation of a heart showing a right-to-left heart shunt;

FIG. 1A is an enlargement of a portion of the schematically illustrated heart of FIG. 1;

FIG. 2 is a schematic diagram showing the interrelationship of the right and left sides of the heart in conjunction with lungs and a right to left shunt;

FIG. 3 shows a representative indicator dilution curve with a preliminary curve indicating a shunt and showing the relative timing of a Valsalva Maneuver along with a time for injecting indicator into a vein;

FIG. 4 is a graph relating pressures between the right and left side of the heart and showing a reversal of such pressures at the point in time of the release of a Valsalva Maneuver;

FIG. 5 is an end view of a reflectance type indicator sensor;

FIG. 6 is a schematic view of a hand positioned over tubing simulating the movement of indicator and normal saline in an artery;

FIG. 7 is a partial sectional view taken through the plane 7-7 shown in FIG. 6;

FIG. 8 is a schematic representation of a reflection mode fluorescence sensor;

FIG. 9 is a perspective view of a bench-testing device utilizing a phantom tissue material;

FIG. 10 is a perspective view of an outer support described in connection with FIG. 9;

FIG. 11 is a perspective view of a phantom material containing carriage mounted in the support of FIG. 9;

FIG. 12 is a front view of the carriage shown in FIG. 11 revealing height adjustment features and laser beam cross sections;

FIG. 13 is a sectional view taken through the plane 13-13 in FIG. 9;

FIG. 14 is a transmission curve showing the performance of an interference filter employed with the instant system;

FIG. 15 is a graph showing the performance of the interference filter with respect to the angle of incidence of photons reaching it;

FIG. 16 is a graph showing measured signal level change with respect to the structure of six collimator plate designs and the utilization of a 1 mm internal diameter tube within a bench test device as described in connection with FIG. 9;

FIG. 17 is a similar chart showing the performance of various aperture structures in conjunction with a 0.5 mm internal diameter tube as utilized in connection with the test rig of FIG. 9;

FIG. 18 is a chart relating ICG concentration with an observed signal level/base-line ratio for transmission as well as reflector mode sensing systems;

FIG. 19 is a schematic view of a human ear showing arterial structure at the scaphoid fossa of such ear;

FIG. 20 is a schematic sectional view of fluorescent excitation and detection at the ear of FIG. 19 utilizing a transmission mode;

FIG. 21A-21G illustrate the structure of a fluorescent sensor intended for use with tissue and arterial structure at the scaphoid fossa of the ear as shown in FIG. 19;

FIG. 22 is a schematic representation of the alignment orientations of three fluorescence generating and sensing devices utilized with the device of FIG. 21A;

FIG. 23 is a stylized curve showing an indicator curve with a recirculation effect and it's relation to a baseline;

FIG. 24 is an animal generated indicator concentration curve showing the recirculation effect;

FIG. 25 is a representation of an indicator dye-dilution curve in conjunction with a preliminary shunt curve;

FIG. 26 shows the curves of FIG. 25 plotted in conjunction with a semi-log graphical representation;

FIG. 27 is a schematic view of the head of a patent with a headband configured to support fluorescence sensing arrays;

FIG. 28 is perspective view of another headband showing two fluorescence array sensors and an electrical cable-receiving hub;

FIG. 29 is a front view of the hub shown in FIG. 28 with the cover removed to reveal internal structure;

FIG. 30 is a perspective view of a monitor, which may be used with the disclosed system;

FIG. 31 is a perspective rear view of the monitor of FIG. 30;

FIG. 32 is a top view of an indicator delivery system;

FIG. 33 is an exploded view of the fluid flow detector utilized in the delivery system of FIG. 32;

FIG. 34 is a side view of the flow detector of FIG. 33;

FIG. 35 is a plan view of a flexible circuit employed with the device in FIG. 34;

FIG. 36 is a sectional view taken through the plane 36-36 shown in FIG. 34;

FIG. 37 is a schematic perspective view of a patient being tested with the disclosed system;

FIG. 38 is a chart showing a human generated Valsalva pressure and dye dilution curves generated at the ears of the patient;

FIG. 39 is a chart summarizing the results of a literature search looking to the transition times used in the past for air bubble contrast or indocyanine green dye indicator;

FIG. 40 is a chart similar to that of FIG. 39 but showing computed transit times and injection positions according to the disclosed system as they are related to a six second Valsalva Maneuver;

FIG. 41 is a chart describing Protocol 1 as utilized with a preferred embodiment of the present disclosure;

FIG. 42 is a chart similar to FIG. 41, but showing a second protocol according to the present disclosure;

FIGS. 43A-43F combine as labeled thereon to show a flow chart of the procedure associated with a preferred embodiment;

FIG. 44 shows one display of a monitor according to the present disclosure as utilized in improving the Valsalva procedure; and

FIG. 45 is a display similar to FIG. 44 but showing a cue to inject indicator.

### DETAILED DESCRIPTION

When a right-to-left shunt is present in the heart or the pulmonary circulation of the human body, in effect a system with two or more alternative blood flow pathways exist. As described above, the most common form of right-to-left shunt in the heart is known as a Patent Foramen Ovale or PFO. During the fetal stage of development, an opening naturally exists between the right and left side of the heart to enable circulation of the mother's oxygen-rich blood throughout the vasculature of the fetus. This opening between the right and left side of the fetus' heart (known as the Foramen Ovale) permanently seals shut in about 80% of the population within the first year following birth. This opening fails to permanently close in the remaining 20% of the population.

For some individuals, this normally closed flap (i.e., Foramen Ovale) temporarily opens allowing blood to flow directly from the right side to the left side of the heart. As a consequence, any blood clots or other metabolically active agents bypass the critical filtering/metabolic functions of the lungs and flow through the brief opening in this flap and directly to the left side of the heart. Once in the left side of the heart, any unfiltered blood clots or agents such as serotonin pass directly into the circulatory system. Since a portion of the blood exiting the left side of the heart flows to the brain as well as the coronary arteries of the heart, any unfiltered blood clots or agents can produce debilitating and life-threatening consequences. These consequences are known to include stroke, heart attack and are also now believed to be one of the principal causes of certain forms of severe migraine headaches.

For further discussion, see the following publications:

9) Spies C., et al., “Patent Foramen Ovale Closure With the Intrasept Occluder: Complete 6-56 Months Follow-Up of 247 Patients After Presumed Paradoxical Embolism,” Catheterization and Cardiovascular Interventions 71: 390-395 (2008);

10) Wammes-van der Heijden E. A., et al., “Right-to-left shunt and migraine: the strength of the relationship,” Cephalalgia; 26: 208-213 (2006);

11) Schwedt T. J., et al., “Patent Foramen Ovale and Migraine—Bringing Closure to the Subject,” Headache 2006 46: 663-671 (2006)

12) Weinberger J., “Stroke and Migraine,” Current Cardiology Reports 2007; 9: 13-(2007).

As disclosed herein, a right-to-left pulmonary shunt is detectable and quantifiable utilizing a biocompatible indicator, which is injected into a peripheral vein of the patient. In connection with this injection, the patient typically is called upon to carry out a Valsalva Maneuver, wherein exhalation into a manometer to achieve a certain pneumatic pressure is called upon for a relatively short interval of time. The release of this maneuver reverses the pressure differential between the right and left atria. The consequence typically is an opening of the noted flap allowing venous blood to flow directly into the left atrium. That flow will be premature with respect to the normal flowpath of venous blood toward the lungs.

The discourse to follow tracks further animal and initial human testing as well as a review of published research is presented resulting in a diagnostic approach which permits a practical survey for the phenomena over a large patient population.

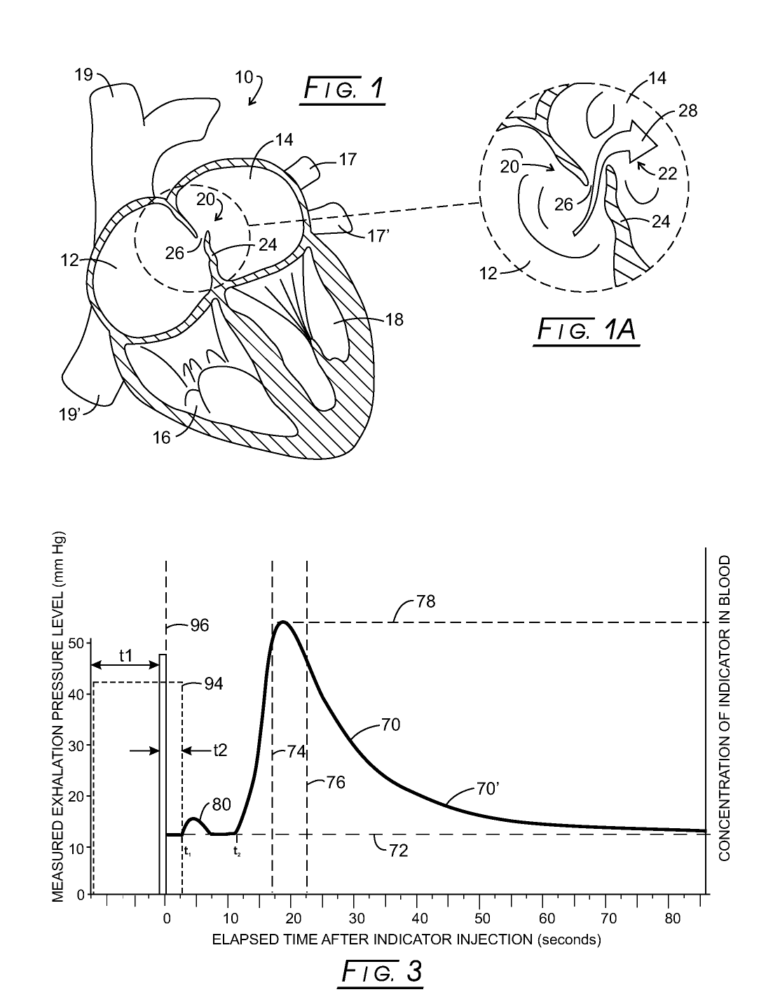

Referring initially to FIG. 1, a mammalian heart is schematically represented and identified in general at 10. The right atrium is shown at 12 and correspondingly, the left atrium is represented at 14. Beneath the right atrium 12 is the right ventricle 16, which is located adjacent to the left ventricle 18. An interauricular septum 20 separates atria 12 and 14 and is shown in FIG. 1A in enlarged fashion to illustrate a PFO represented generally at 22. Typically, venous blood enters the heart through the superior vena cava and inferior vena cava 19 and 19′ feeding the right atrium to the right ventricular and pulmonary artery passing to the lungs. From the lungs, the left atrium is supplied with oxygenated blood via the pulmonary veins 17 and 17′, with that blood then being pumped throughout the arterial system by the left ventricle 18 to the aorta (not shown in FIG. 1). As illustrated in FIG. 1, the atypical presence of a patent foramen ovale 22 results from, for example, the presence of displaceable tissue flap 24, creating opening 26. Shunt flow of venous blood from the right to left atria through opening 26 is represented in FIG. 1A by the arrow 28. Shunt flow thus does not pass through the lungs, bypassing the pulmonary circulatory circuit, and potentially allowing detrimental blood components to bypass the filtering capabilities of the lung capillary beds.

In general, the preferred embodiments of the present disclosure, observes that an indicator such as an externally detectable indicator dye material will traverse through the venous system toward the right atrium within a detectable transit time. Accordingly, venous blood containing such an indicator, will pass the opening 26 between the right and left atria, and progress through the arterial system ahead of indicator carried through the normal circulatory system, i.e., through the lungs.

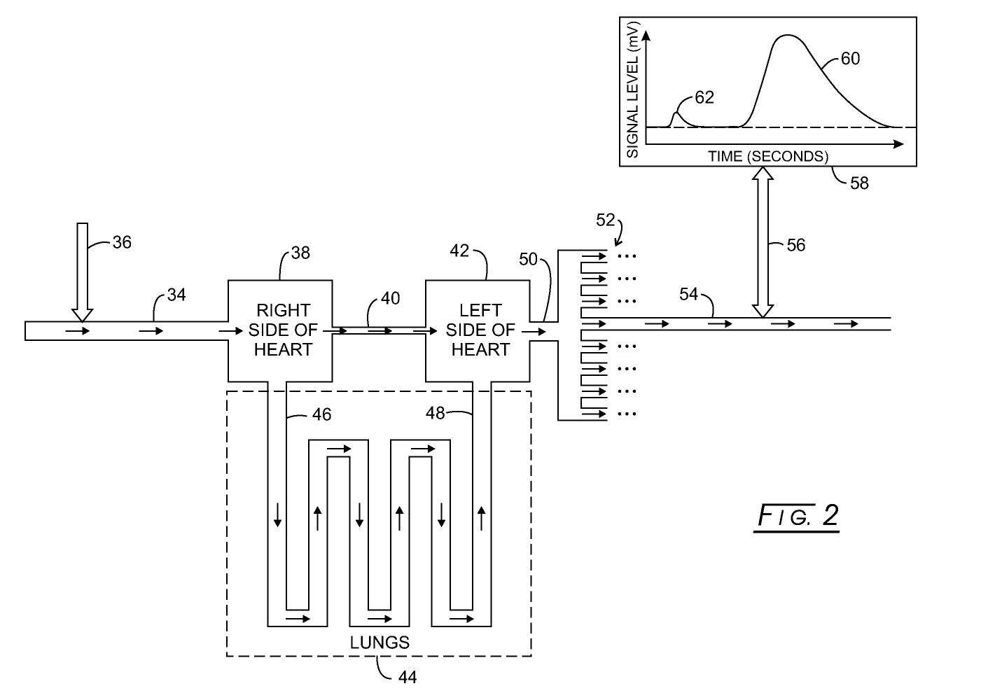

Looking to FIG. 2, such an indicator bolus premature condition and delay by spacing through the lungs is represented schematically. In the figure, indicator being introduced to the venous blood stream is represented at arrow 36. Preferably, arrow 36 represents the injection of a predetermined amount of indicator in a peripheral vein, i.e., the antecubital vein in the right arm of a patient. This is followed by an injection of isotonic saline. The indicator in venous blood at blood stream 34 is directed to the right side of the heart as represented at block 38. A right-to-left shunt is represented by the small conduit 40, which is shown extending to the left side of the heart as represented at block 42. Meanwhile, the lungs are represented within the dashed boundary 44, a circuitous route of filtering and aeration being represented by conduit 46 as it extends from the right side of the heart to conduit 48. As it travels from the right side of the heart following filtering and aeration, the refreshed blood now enters the left side of the heart as at block 42, whereupon it is distributed as represented at conduit 50. From conduit 50 the refreshed blood is distributed to multiple arterial conduits represented in general at 52. One conduit of the conduit array 52 is seen at 54 being analyzed by the sensor and controller function of the instant system represented by arrow 56. Other such arterial conduits may be simultaneously analyzed to provide a plurality of sensing outputs, which are time and intensity based dilution curves, for example, generated by a dye indicator. Such dilution curves based upon signal intensity level and time are represented schematically at 58, only one such curve being shown. These curves at display 58 result from the intensity of the indicator and its transit time from injection as represented at arrow 36. The principle of the dilution curve at display 58 is that it is the detection of an indicator bolus resulting from the passage of indicator 60. However, note that a premature and smaller indicator detection and dilution curve 62 is present which results from the passage of indicator along the shunt 40. Curve 62, representing, for example, a PFO can be quantified by a ratiometric analysis, with reference to dilution curve 60. Thus, not only is the presence of a PFO detected but it is quantified. Any recirculation component of the indicator will have been removed from the principal curves as at 60 and 62. It is possible that more than one of the premature curves as at 62 can occur.

Referring to FIG. 3, a stylized representation of the indicator dilution curves and associated procedures for their use is stylistically presented. In the figure, a principal dilution curve representing indicator passage through the lungs is represented at larger curve 70. Idealized curve 70 is shown commencing at a time represented at t₂, and is shown to reside just above a baseline represented at horizontal dashed line 72. The peak of curve 70 is shown to reside between vertical dashed lines 74 and 76 and exhibits a peak indicator concentration at horizontal dashed line 78. The descending component of curve 70 as shown at 70′ is calculated to accommodate for recirculation phenomena and the like.

Occurring prior to curve 70 is a preliminary curve 80 commencing at time, t₁, and representing a pulmonary shunt condition, which can be quantified with respect to curve 70.

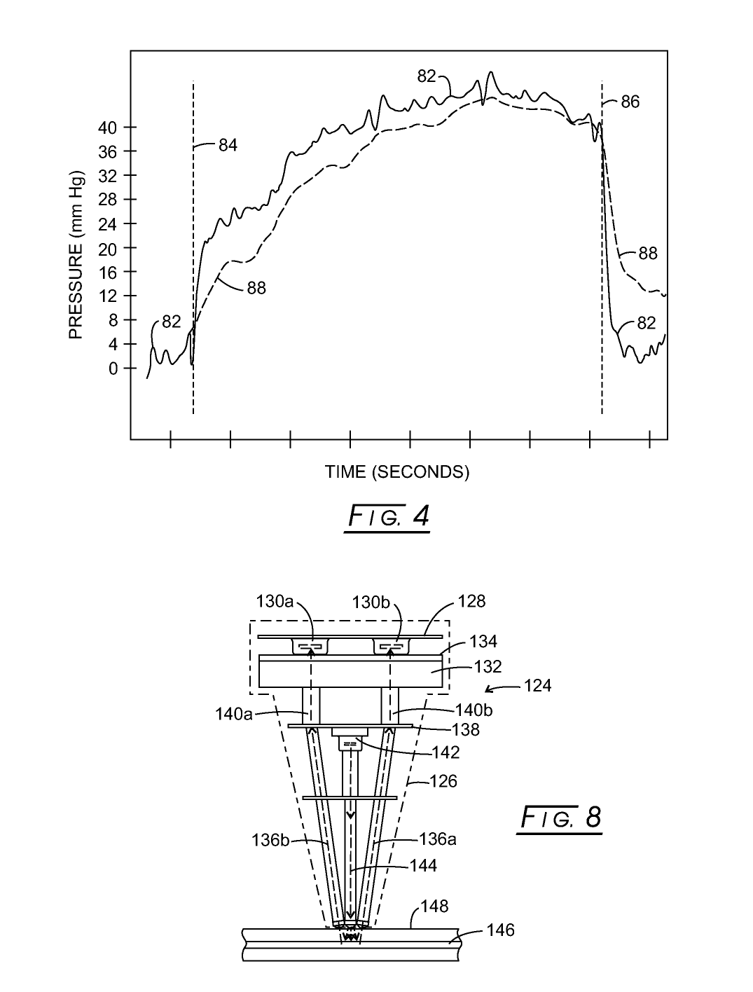

Returning momentarily to FIG. 1, under normal pulmonary conditions, the opening 26 in the heart will be closed, for example, by flap 24 and the presence of a differential pressure having a higher level in the left atrium 14. This pressure differential can be reversed by applying and releasing an exhalation pressure, for example, between 30 and 45 milliliters of mercury. Looking momentarily to FIG. 4, the pressure differential between the left side of the heart and the right side of the heart is plotted with respect to time. In the figure, measured pressure on the left side of the heart (PCWP) is shown at plot 82 extending in time during a Valsalva Maneuver, starting at vertical dashed line 84 and ending with release represented at dashed line 86. Pressure on the right side of the heart (RAP) is represented at plot 88. Note that initially at the commencement of the Valsalva Maneuver, pressure on the right side of the heart is lower than that on the left. However, at the release of the Valsalva Maneuver as represented at dashed vertical line 86, the differential pressure between the left and right side of the heart reverses, pressure on the left side of the heart being lower than that on the right. This will tend to open any flap-type valving as shown in FIG. 1 at opening 26. (See: Pfleger 2001).

Returning to FIG. 3, as is apparent, the timing of the Valsalva Maneuver as well as the injection of indicator are important components of the instant system. In the figure, the Valsalva Maneuver with respect to curves 70 and 80 is represented within the dashed boundary 94 showing the release of the Valsalva Maneuver at time, t₁. The time of injection of the indicator is represented by the bar and dashed line 96, the figure showing injection commencing at time, t₁, and the time between the commencement of injection and release of the Valsalva Maneuver being represented as a time, t₂.

Now looking to the indicator, a circulatory tracking reagent is called for. Studies at the outset of the research leading to the present invention a preferred embodiment was to employ fluorescing dyes, certain of which had been approved for use in humans. Two such exemplary dies were available at the time of the study, fluorescein and indocyanine green dye (ICG). The latter indicator was elected.

A number of additional circulatory tracking reagents are available for use with the system that had, including such indicators as follows: U.S. Pat. No. 3,412,728 describes the method and apparatus for monitoring blood pressure, utilizing an ear oximeter clamped to the ear to measure blood oxygen saturation using photocells which respond to red and infrared light. U.S. Pat. No. 3,628,525 describes an apparatus for transmitting light through body tissue for purposes of measuring blood oxygen level. U.S. Pat. No. 4,006,015 describes a method and apparatus for measuring oxygen saturation by transmission of light through tissue of the ear or forehead. U.S. Pat. No. 4,417,588 describes a method and apparatus for measuring cardiac output using injection of indicator at a known volume and temperature and monitoring temperature of blood downstream. This and several similar systems in the art suffer from an inability to effectively quantify the magnitude, i.e., functional conductance of shunts as opposed to the presently disclosed embodiments.

A number of patents describe potential reagent systems that if adapted could be utilized with the present system method and apparatus. U.S. Pat. No. 4,804,623 describes a spectral photometric method used for quantitatively determining concentration of a dilute component in an environment (e.g., blood) containing the dilute component where the dilute component is selected from a group including corporeal tissue, tissue components, enzymes, metabolites, substrates, waste products, poisons, glucose, hemoglobin, oxy-hemoglobin, and cytochrome. The corporeal environment described includes the head, fingers, hands, toes, feet and ear lobes. Electromagnetic radiation is utilized including infrared radiation have a wavelength in the range of 700-1400 nanometers. U.S. Pat. No. 6,526,309 describes an optical method and system for transcranial in vivo examination of brain tissue (e.g., for purposes of detecting bleeding in the brain and changes in intracranial pressure), including the use of a contrast agent to create image data of the examined brain tissue.

Looking to the indocyanine green dye (ICG), excitation curves have been illustrated as having a peak excitation wavelength at about 785 nanometers. Correspondingly, for the fluorescent emission of the two fluorescent dyes, a peak wavelength of fluorescing photons resides at about 830 nanometers.

To use this fluorescing form of indicator in carrying out pulmonary shunt detection and quantification, a sensor having the capability to direct laser excitation illumination to a blood vessel as well as to collect and filter an emitted fluorescent response, sensors were developed. The sensors so developed operate either in a reflection mode or a transmission mode.

In initial studies the reflective mode was utilized for the sensor. A relatively simple sensor was evolved utilizing fiberoptic technology. Looking to FIG. 5, one end of such a sensor 100 is revealed. At the center of sensor 100 is a fiberoptic channel identified at 102, which projects excitation emissions, for example, at 785 nanometers for ICG. Surrounding central fiber 102 are seven glass fibers identified at 104a-104g. All of these glass fibers have an outside diameter of, for instance, about 600 microns. When an ICG indicator within the bloodstream reaches the site of irradiation with 785 nm prime laser (light), the fluorescent moiety within the ICG indicator is excited to an elevated energy state for a brief period. As the excited moiety returns to its normal energy state, it emits light at a longer wavelength (e.g., 830 nm), and the difference between the excitation wavelength (785 nm) and the fluorescence emission wavelength (vis 830 nm) is known as the Stokes Shift. This Stokes Shift of nominally 45 nm allows the fluorescent emission to be extracted by using a wavelength band of interest (vis 820-840 nm).

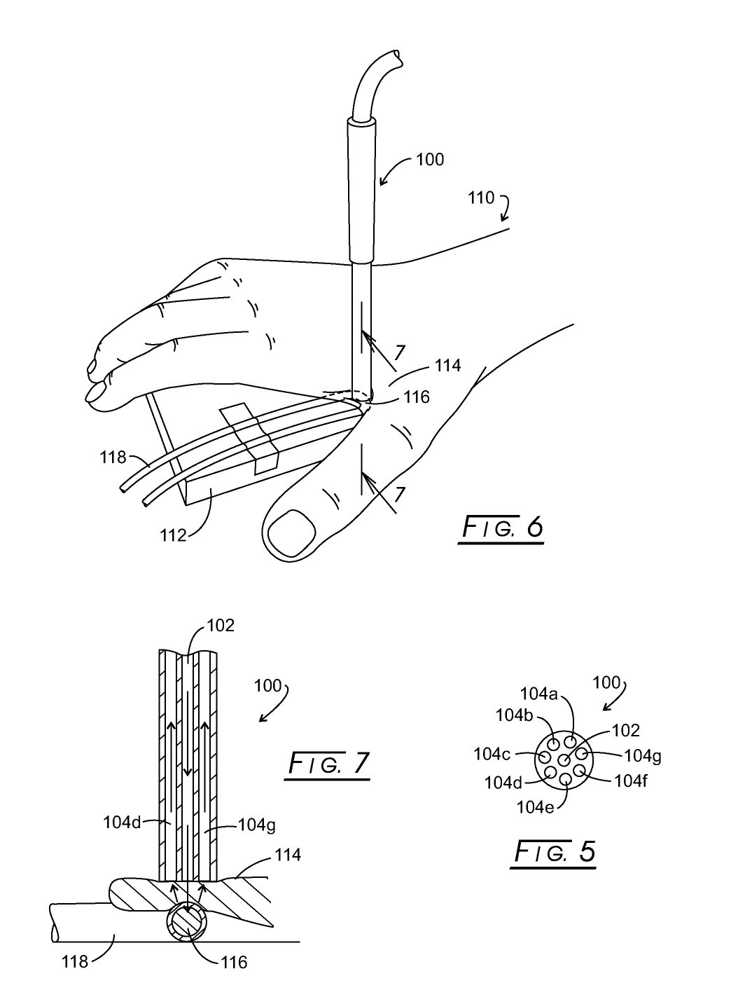

Such a reflection mode sensor was used initially in a bench top test determine the light scattering influence of a thin human tissue. Looking to FIG. 6, a human hand is represented at 110 positioned upon a bench 112 with the tip of sensor 100 being located against the web portion 114 of such hand. That web portion 114 is seen to be positioned over the curve region 116 of a transparent tube represented generally at 118. In general, the test represented at FIGS. 6 and 7 sought to evaluate the light scattering characteristics of a thin portion of human tissue. In this regard, varying dosages of ICG followed by clear water were passed through tube 118. As seen in FIG. 7, laser excitation light is directed downwardly through sensor fiberoptic 102 to pass through the skin region 114 and into the material within tube 118. This causes a fluorescence to enter the outside fiberoptic components as represented by upwardly pointing arrows. The results of this testing were used to develop a bench top phantom test rig.

Referring to FIG. 8, a design for a reflection mode sensor is presented. Represented generally at 124, the conically-shaped sensor is shown having a tapered aluminum body seen in phantom at 126 which surmounts a circuit board 128 supporting three photodiodes, two of which are seen at 130a and 130b. These photodiodes are positioned above an interference filter 132, which encounters and passes 830 nm fluorescence generated photons. Above the filter 132 is a blocking filter for 785 nm excitation photons as shown at 134. The fluorescing photons are collected by optical fibers, two of which are shown at 136a and 136b. These fibers extend to lenses (not shown) intended for focusing wide-angle fluorescent photons into the fibers 136a,b. These fibers extend through a platform 138 to pass the photons through collimating lenses as at 140a-140b. Coupled to the underside of platform 138 is a combination of a laser diode (785 nm) and collimating lens 142. The collimating lens combination 142 feeds red region laser light energy to a laser fiber 144 which extends to excite the indicator which may be carried within a blood vessel 146 located within tissue 148.

Sensors performing in a transmission mode as opposed to a reflection mode were developed in conjunction with a tissue phantom holder designed for bench top experimentation and analysis.

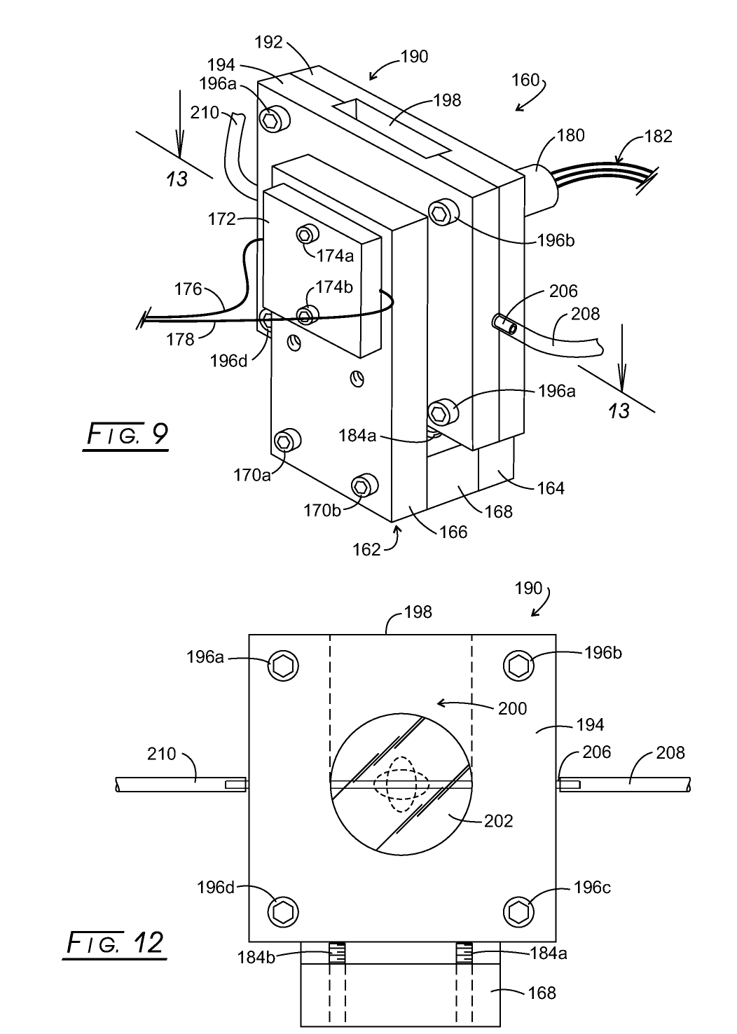

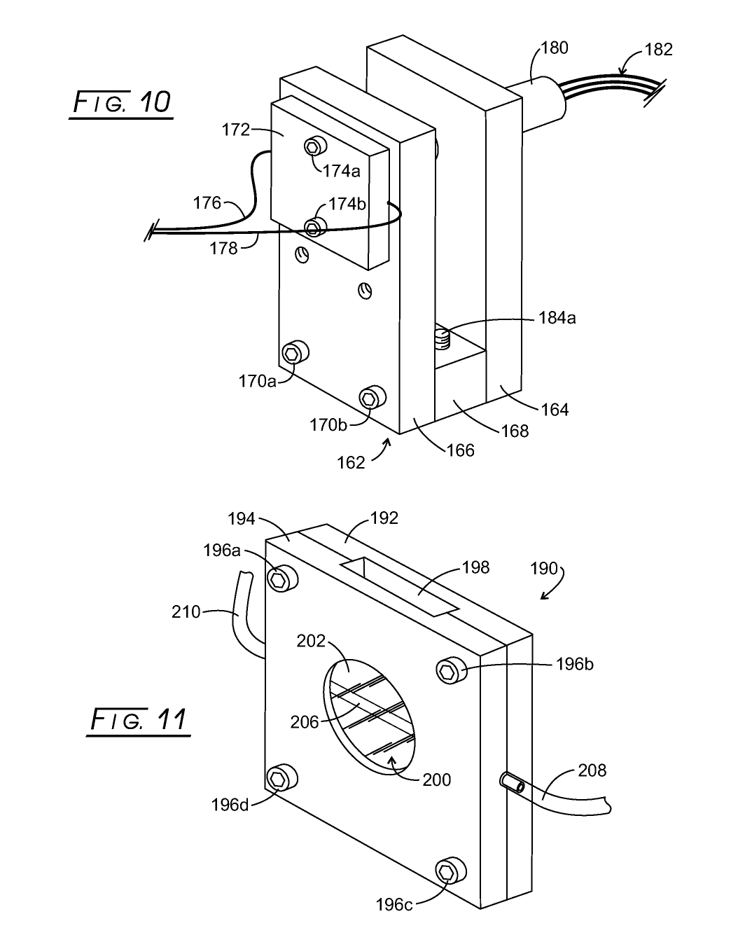

Referring to FIG. 9, the test apparatus used for bench top testing is represented in general at 160. Apparatus 160 includes a generally U-shaped optic support shown generally at 162 which supports a laser diode at a front face 164 and a spaced apart back face 166 supporting a photodiode. The spacing and support of faces 164 and 166 is by a base plate 168. Note the attachment of plate 166 with faceplate 168 by cap screws at 170a and 170b. Back face 166 supports a photodiode assembly which is retained in place by a retainer block 172, attached to plate 166 by cap screws 174a and 174b. Electrical leads extending to the photodiode are represented at 176 and 178. Front face 164 supports a laser diode assembly (not shown) which is supported electrically and mechanically by a laser diode retainer 180 shown having electrical leads extending therefrom in general at 182. U-shaped subassembly is shown in isolation in FIG. 10. It may be seen that one of two adjustment screws is present at 184a.

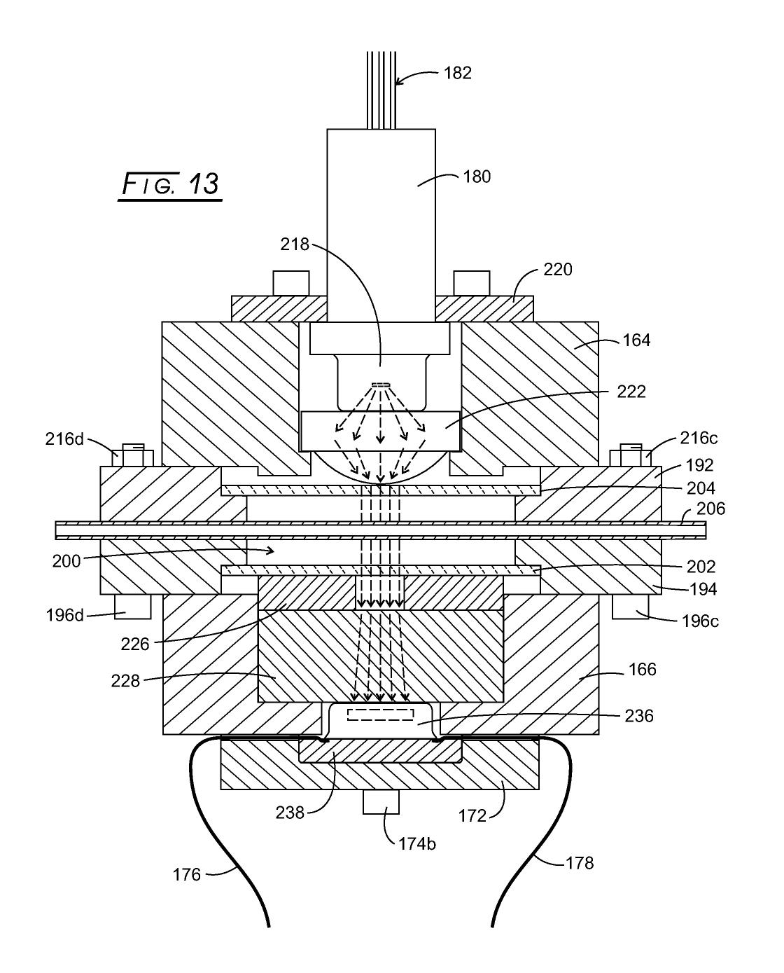

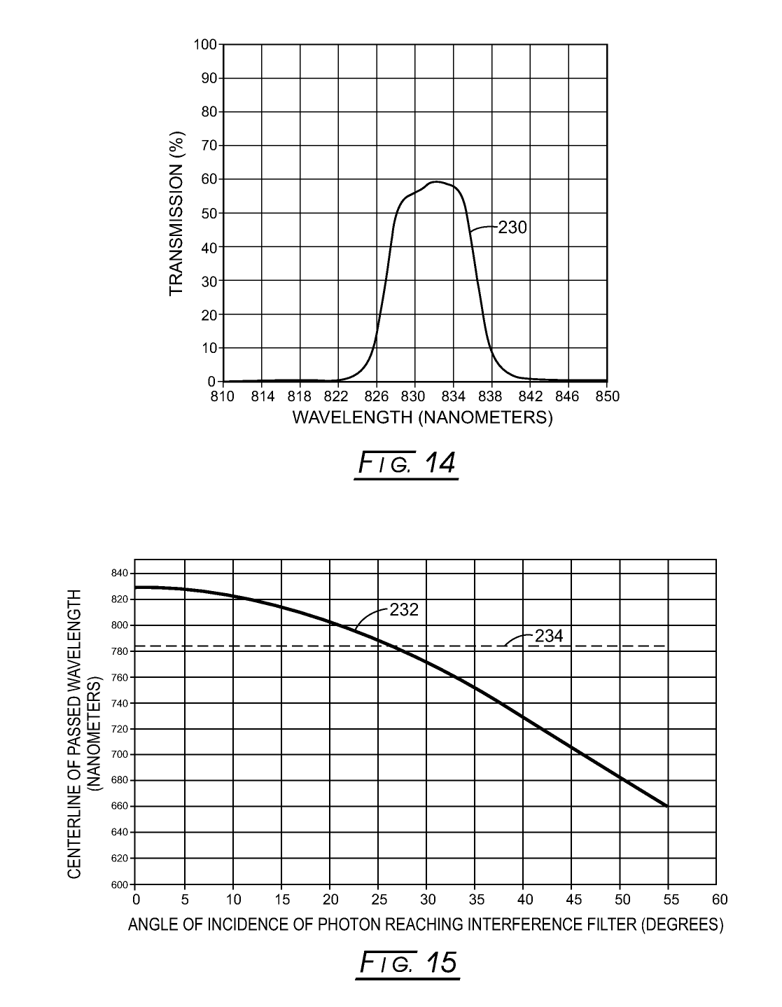

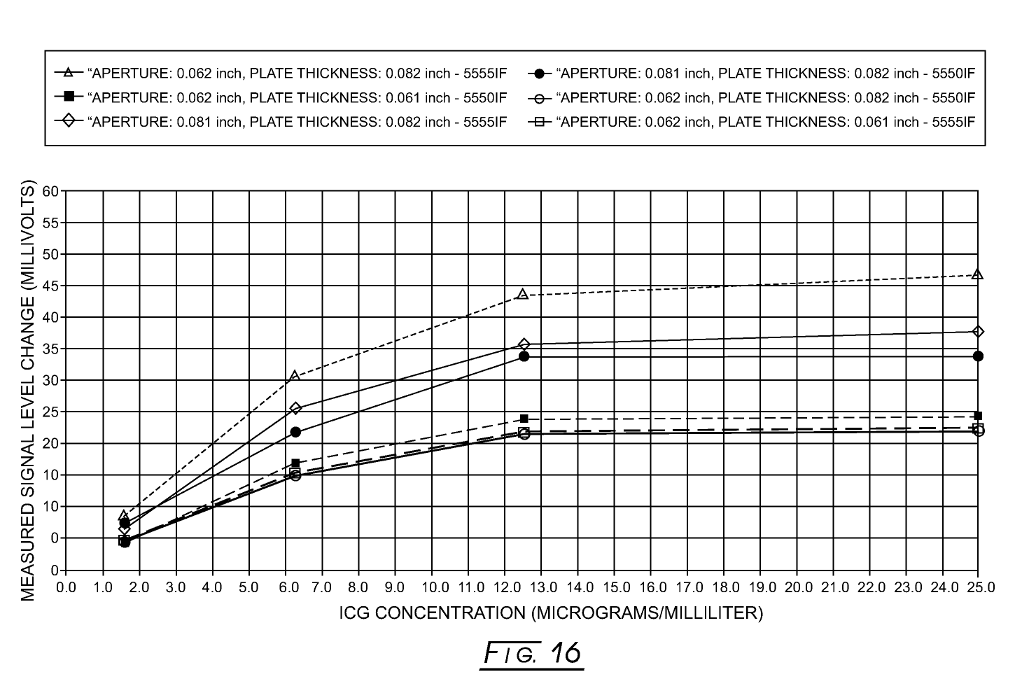

Between the faceplates 164 and 166 there rides a phantom carriage represented generally at 190. Carriage 190 is formed of two plates, 192 and 194 which are held together by four bolt and nut assemblies, the bolts of which are shown at 196a-196b. Plates 192 and 194 are joined together to form a phantom tissue defining cavity having an uppermost slot accessing entrance at 198. Looking additionally to FIGS. 11 and 12, the cavity is represented generally at 200 and is surmounted by two circular windows, one of which is shown at 202 within plate 194. Cavity 200 is configured to retain a tissue emulating material marketed under the trade designation “Intralipid” which is adjusted to emulate human tissue having a thickness of about 3 mm. The tissue characteristic emulated is derived from the experimentation of FIG. 6. During that test, tubes of 1 mm and 0.5 mm were used with varying concentrations of ICG. Extending through the cavity 200 is a glass tube 206, having an internal diameter, for example, of 0.5 mm or 1 mm, emulating the size of a blood vessel at the ear pinna. Tube 206 is connected for fluid input and return by flexible tubing 208 and 210. FIG. 12 reveals that the phantom carriage 190 is adjustable vertically by adjusting screws 184a and 184b. The optics within faceplate 162, as well as the phantom carriage 190, cooperatively perform in a transmission mode wherein laser energy is projected through one side of the skin (here tissue emulating material), and resulting fluorescing photons are detected on the opposite side of the tissue component being examined. This is revealed in the sectional presentation of FIG. 13. In that figure, cavity 200 reappears in conjunction with circular windows 202 and 204 formed within respective carriage plates 194 and 192. Nuts 216c and 216d are seen to be threadably attached to respective bolts 196c and 196d. A laser diode 218 is seen coupled with retainer 180 and is retained in position within plate 164 by a ring retainer 220. Positioned to intercept and collimate photons from the infrared diode 218 is a collimating aspheric lens 222, the resulting collimating photons being represented in general at 224, impinging upon the glass tube 206. Laser diode 218 may, for example, be a type DL7140-201S(785MM), marketed by Tottori SANYO Electric Ltd of Tachikawa, Japan.

Returning momentarily to FIG. 12, it may be observed that the beam resulting from the combination of laser diode 218 and aspheric collimating lens 222 has an elliptical cross-section. Tests were made with the larger diode being turned 90° about its axis and the resultant elliptical cross-sectional beams are shown in FIG. 12. It was determined that the orientation of the beam with respect to that cross section was of no importance.

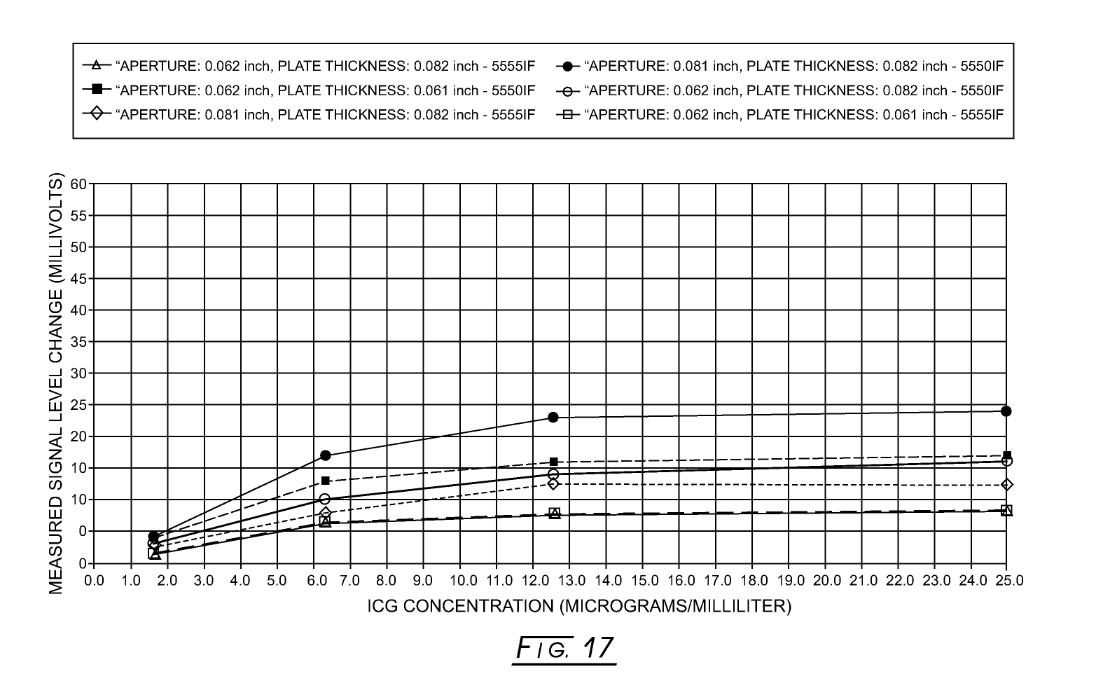

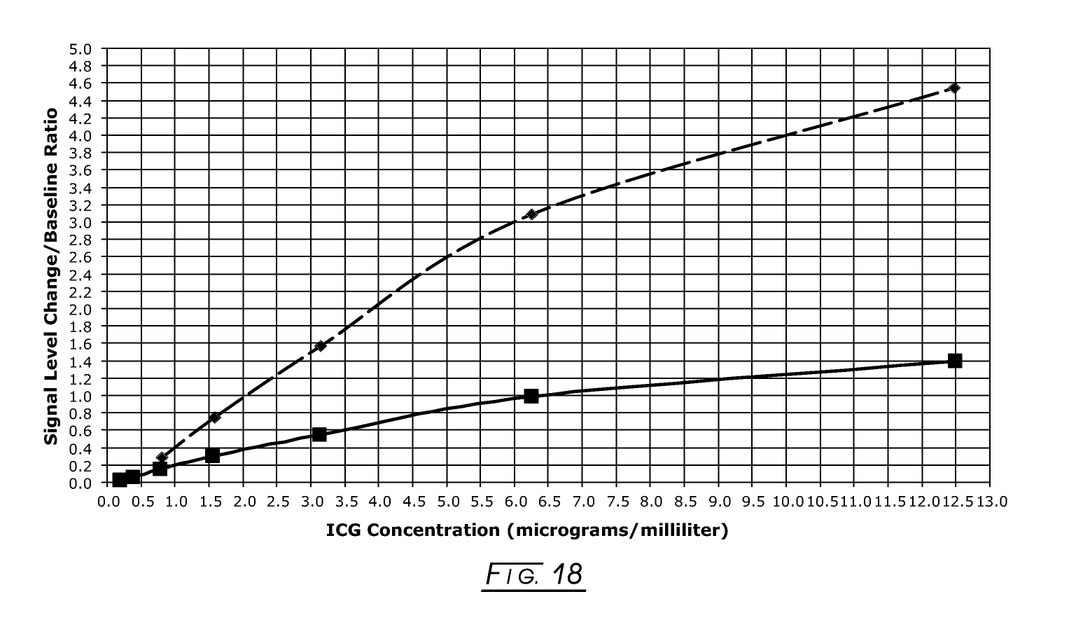

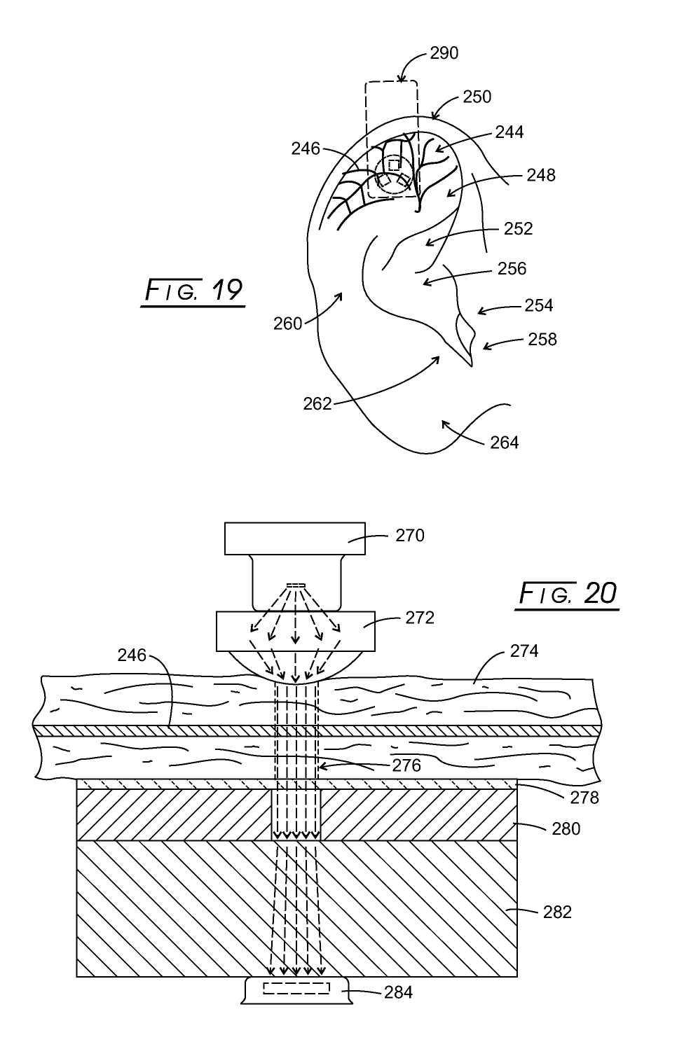

Returning to FIG. 13, fluorescing photons having a wavelength of about 835 nm are caused to pass through an aperture of an opaque collimator (collimator plate) 226 from which they encounter an interference filter 228. The performance of filter 228 is represented at band pass curve 230 shown in FIG. 14. The structure of the collimator 226 was developed using the bench top assembly 160, as will be discussed in relation to FIG. 16. Looking additionally to FIG. 15, the performance of interference filter 228 is represented at curve 232 which reveals that its performance is dependent upon the angle of incidence of photons reaching it. For example, from curve 232 one may observe that for a zero angle of incidence, a full 835 nm wavelength photon will pass. Performance degrades as that angle of incidence increases. The output of the laser 218 is shown at dashed line 234.

Returning to FIG. 13, a photodiode is represented at 236 along with earlier-described leads 176 and 178. Photodiode 236 may be a type DPW34BS, marketed by OSRAM. The device at 236 is retained in position by retainer block 172 and a foam insert 238.

Referring to FIGS. 16 and 17, benchtop tests of various collimating plates as at 226 for a transmission mode of performance, in combination with two different interference filters. In FIG. 16, the tests were performed utilizing a glass tube 206 having an internal diameter of 1 mm. The curves represent a measured signal increase between water and ICG using a 4.5% Intralipid tissue phantom. By contrast, FIG. 17 shows the same test carried out but with a glass tube 206 having an internal diameter of 0.5 mm. A preferred embodiment is utilizing a collimator plate with an aperture of approximately 0.081 inch and a plate thickness of 0.082 inches and the 5550 interference filter, which provided results consistent between the 1 mm and 0.5 mm glass tubes.

The transmission mode of sensing as described in connection with FIG. 13 finds advantageous application at regions of the body in which surface tissues are relatively thin. The transmission mode of sensing is preferably applied to locations of the patient body wherein the arterial vasculature is arranged such that the transmissive sensors can be placed opposite the photodetectors in a noninvasive manner. Preferred locations on the human body include the pinna of the ear, the hand, including the web of skin between the thumb and forefinger, the neck, including distendable skin about the neck, the leg, and the arm, including distendable skin of the arm proximal to the shoulder. A preferred embodiment of the system is to place sensor arrays at symmetrically paired locations distal to the heart, such as at is both ears, both hands, paired locations on the neck, the leg, and the arm.

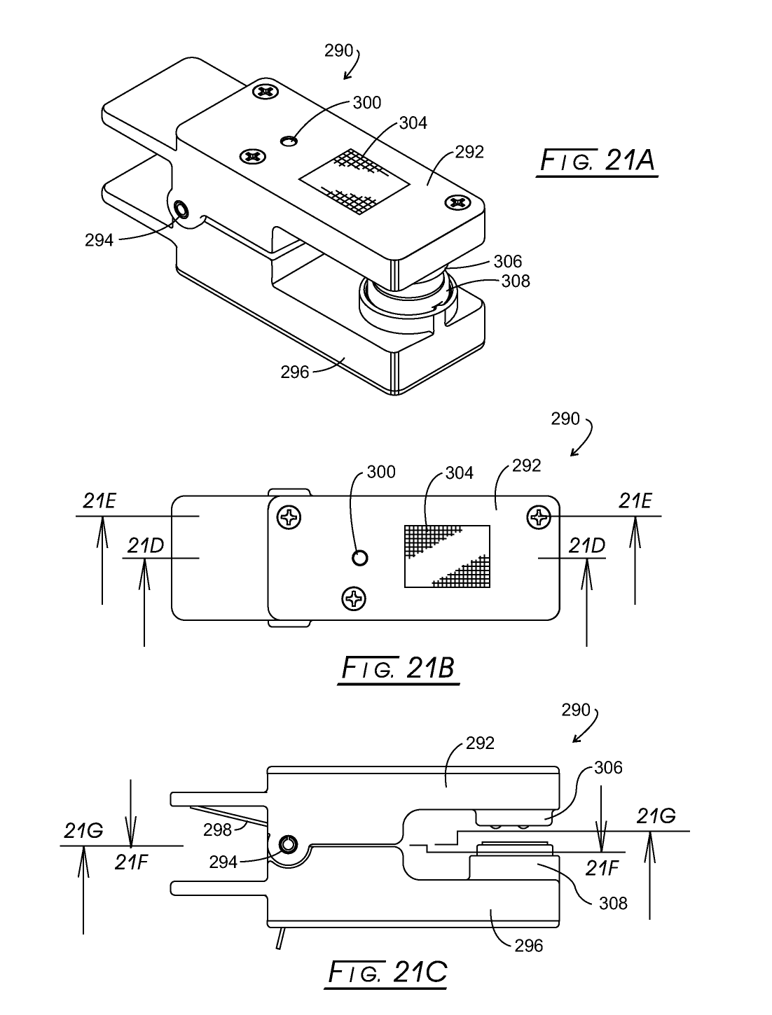

A particularly preferred embodiment is placement of sensor arrays on both pinna of the ears of the human patient. Looking to FIG. 19, the human ear pinna is revealed in conjunction with the outline of the transmission mode sensor having three laser driven transmissive sensors arranged at that portion of the ear termed herein as the scaphoid fossa and identified generally at 244. Arterial vessels are shown in this region as lines 246. Further regions of the ear are identified as the triangular fossa shown generally at 248; the helix shown generally at 250; the concha is shown generally at 252; the tragus shown generally at 254; acoustic meatus shown generally at 256; the intertragic notch shown generally at 258; the anti helix shown generally at 260; the anti tragus shown generally 262; and the lobule shown generally at 264. For a more detailed discussion of the ear see: Tilotta, F, et al., Surg. Radiol. Anat., 31:259:265 (2009).

Turning now to FIG. 20, a portion of the scaphoid fossa again is identified at 244 in conjunction with an arterial vessel as earlier described at 246. A transmission mode of sensing is shown associated with that part of the ear. The components of the transmission mode sensor include a laser diode 270, the output of which is associated with an aspheric collimating lens 272. Direct laser light as represented at 274 into the scaphoid fossa 244 to impinge upon arterial vessel 246. Laser light and fluorescence generated photons then occur as represented in general at 276, to pass a transparent window 278 the bore of an opaque collimator 280 and interference filter 282. Filter 282 passes essentially only the photons resulting from fluorescence to impinge upon a photodetector 284.

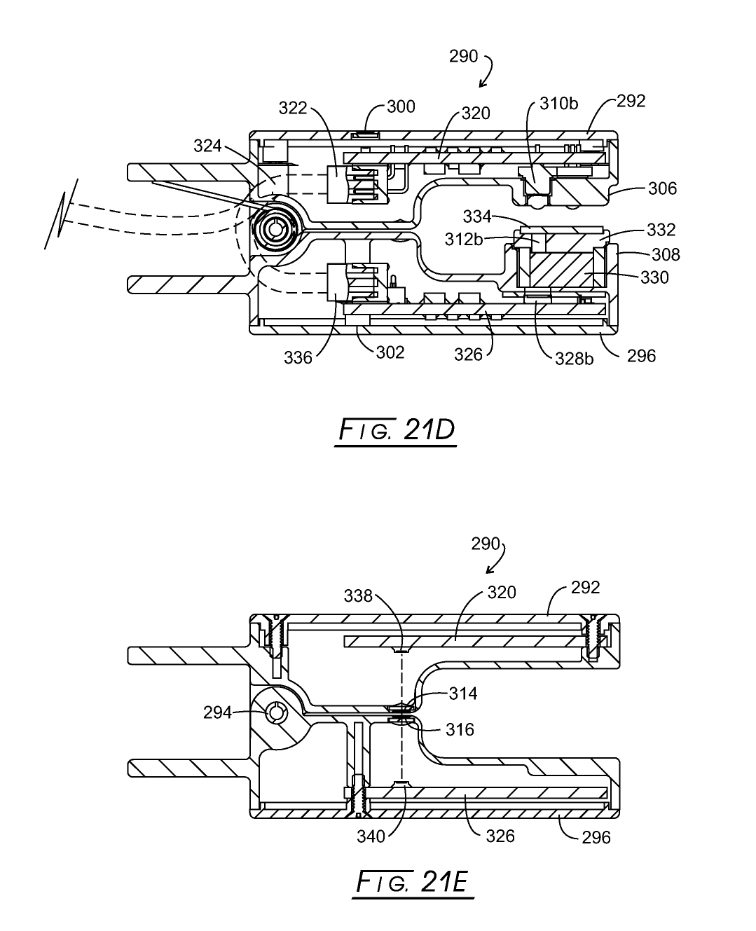

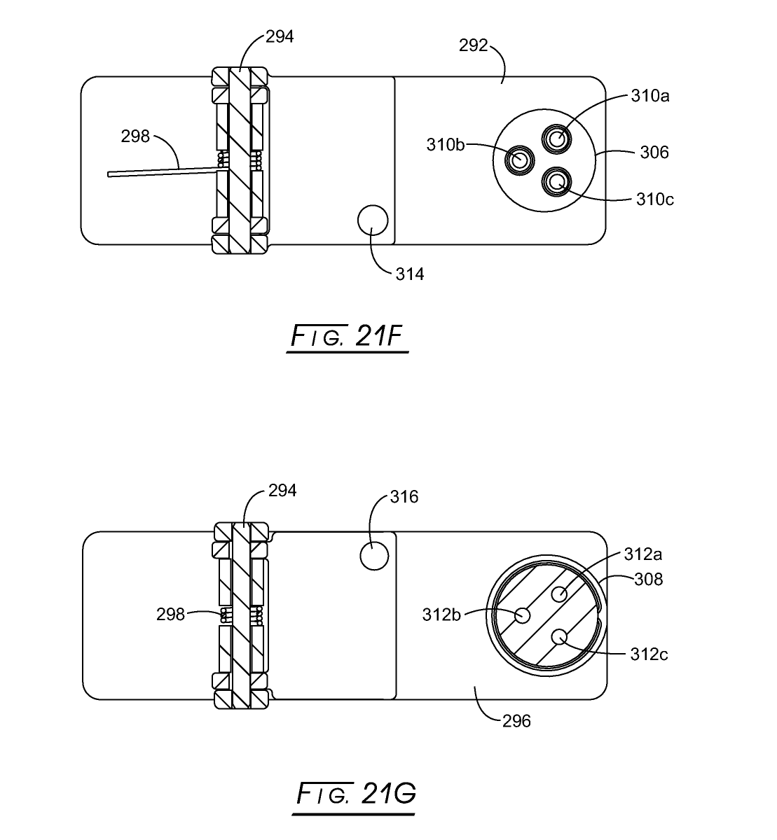

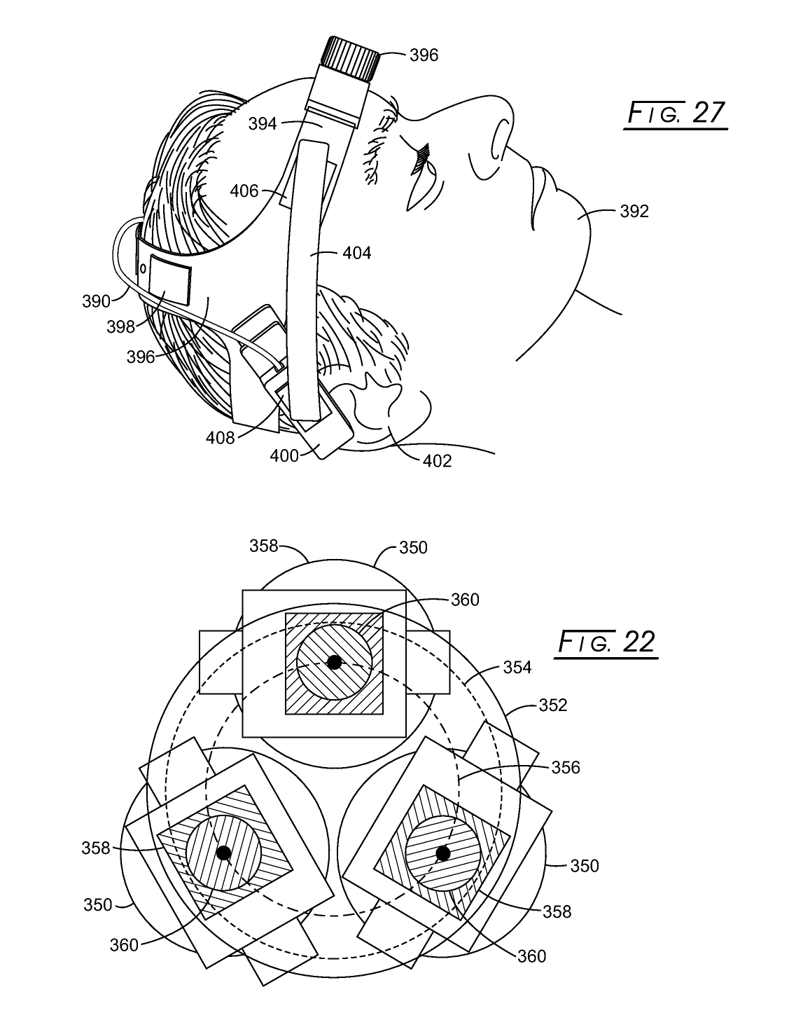

The component described in connection with FIG. 20 are implemented with a sensing array fixture shown in perspective and identified in general at 290 in FIG. 21A. Fixture 290 is formed with a three laser array support 292 which is hinged at 294 to a photodiode array support 296. Supports 292 and 296 are biased toward each other by a spring seen in FIG. 21C at 298. A laser “power-on” light emitting diode provides a yellow colored light output at 300. As seen in FIG. 21D, a similar yellow LED is located in support 296. It has been found to be beneficial to incorporate a Velcro-type pad for the support of device 290. Such a pad is represented at 304. A three laser array along with collimating aspheric lenses is mounted within a protrusion 306 extending inwardly from support 292. That protrusion is seen, particular, at FIGS. 21C, 21D and 21F. Complementing the three laser array is an aligned array of three photodiodes located within protrusion 308 which also is seen in FIGS. 21C, 21D and 21G. Looking to FIG. 21F, protrusion 306 is seen to support an array of three lasers and associated aspheric collimating lenses as represented at 310a-310c, aligned with those laser and collimating lens as are corresponding photodiodes along with an associated collimator and interference filter. The collimator openings for orifices are seen at 21G at 312-312c. Also seen in FIGS. 21F and 21G are windows 314 and 316, utilized in providing a laser interlock system.

Looking to FIG. 21D a sectional view is shown through the section identified at 21B-21D in FIG. 21B. The figure shows that a circuit board 320 is mounted in support 292 which is shown supporting a laser diode and an aspherical lens 310b, as well as cable connector 322 intended for connection with a cable component 324. Circuit board 320 additionally supports laser diodes 310a and 310c, diode 310b being seen in this sectional view.

In similar fashion, support 296 incorporates a circuit board 326 which supports three photodiodes, one of which is seen at 328d, located beneath interference filter 330, collimator 332 having orifice 312b and a transparent window 334. Circuit board 326 also incorporates a cable connector 336 which also is coupled to cable 324. A laser power-on LED is shown at 302 as well as at 300.

Referring to FIG. 21E, a section of device 290 is shown taken through the plane 21E-21E shown in FIG. 21B. In the figure, circuit boards 320 and 326 reappear, board 320 supporting a light emitting diode 338 which operationally performs with an aligned photodetector 340, light from the LED 338 extending through the windows 314 and 316 to excite the photo detector 340 and provide an optical interlock having a signal utilized by the control circuitry.

Referring to FIG. 22, an alignment diagram shows the relative positioning of the components of the fluorescence sensor array employed with devices as at 290. In the figure, the physical diameter of the laser diodes is represented at 350. These devices are identified as Sanyo Laser Diodes, catalog number DL-7140-201S that have a 0.220 inch diameter. Circle 352 represents the outer diameter of an unmounted Edmund Optics interference filter, while circle 354 represents the clear aperture of a mounted Optasigma interference filter. Circle 356 represents the center-line of the laser diodes and photo diodes. Squares 358 represents the active area of the photodetectors marketed by Osram and circles 360 represent the elliptical cross-section of the laser beams.

Referring to FIG. 23, a theoretical dye dilution curve is represented at 366 in conjunction with a baseline 368. In order to compute the area under such curves, account must be made of the recirculation effect. That effect is represented by the dashed curve 370. In general, the controller circuitry used with the system will compute the exponential decay shown as solid line region 372, whereupon area under the curve represented at 366 and 372 may be computed.

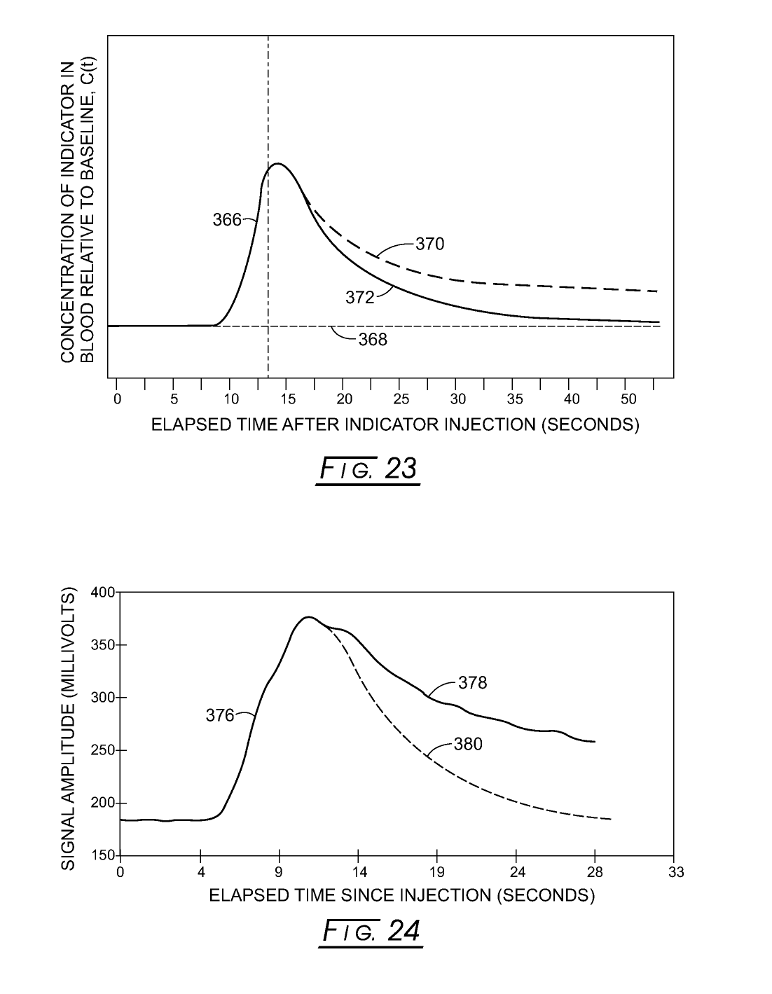

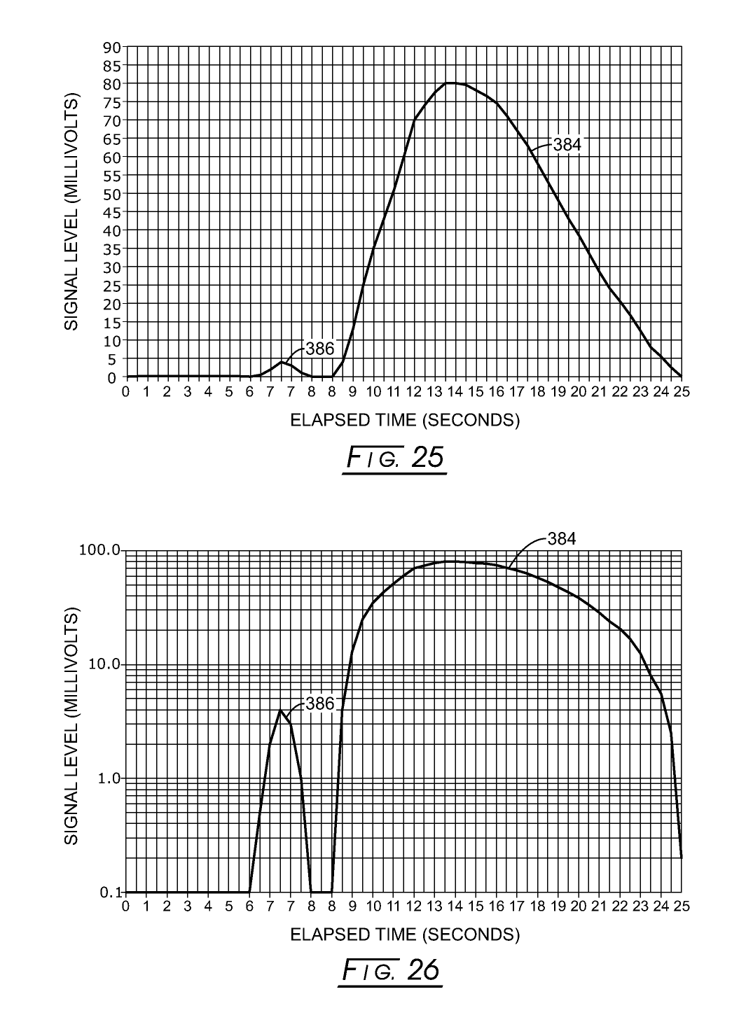

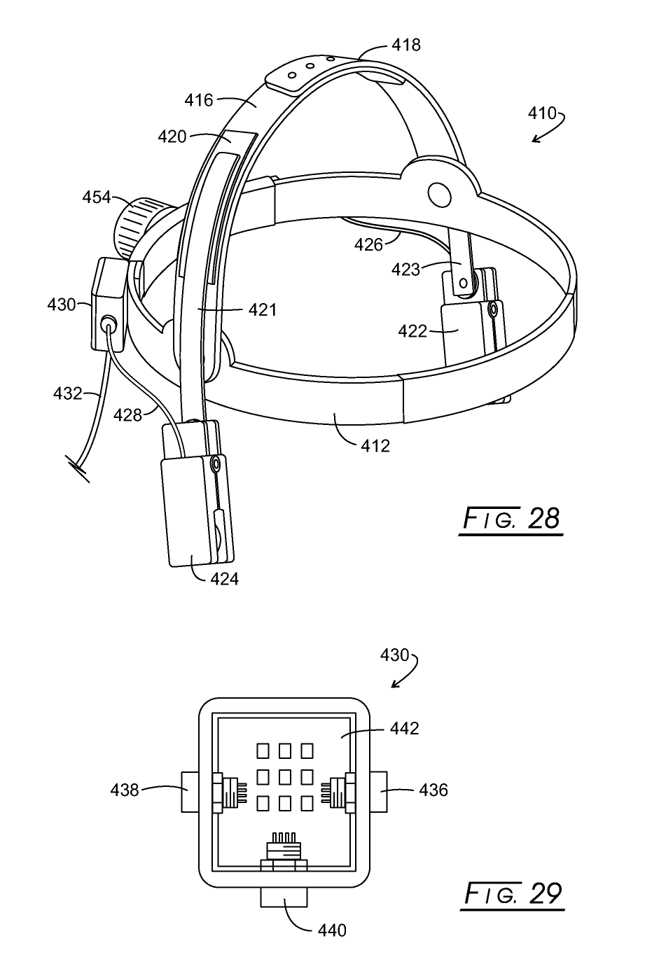

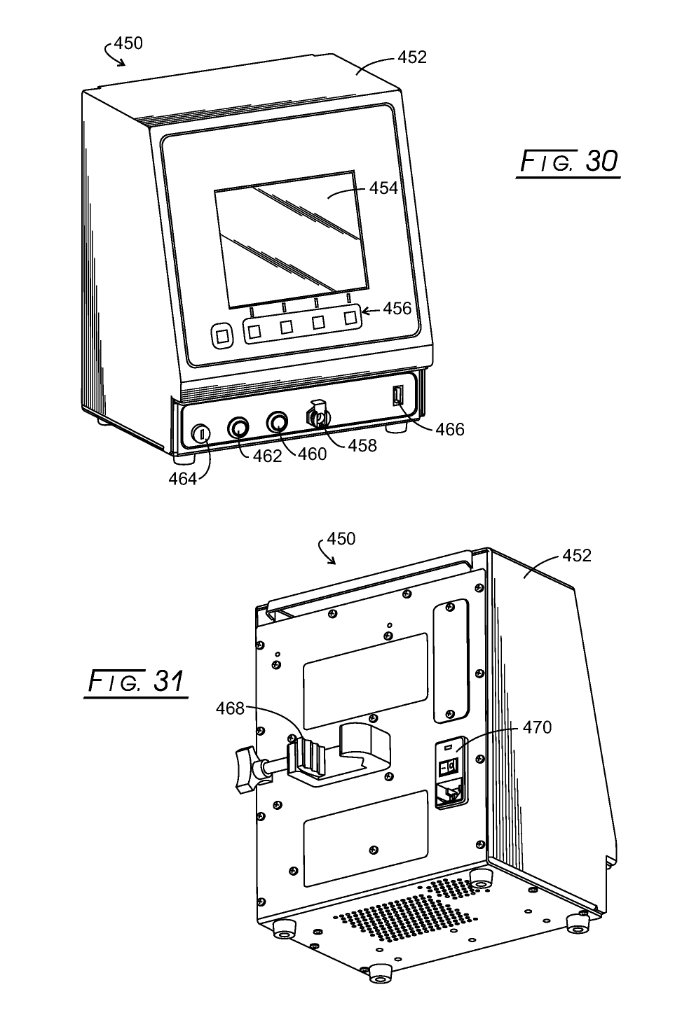

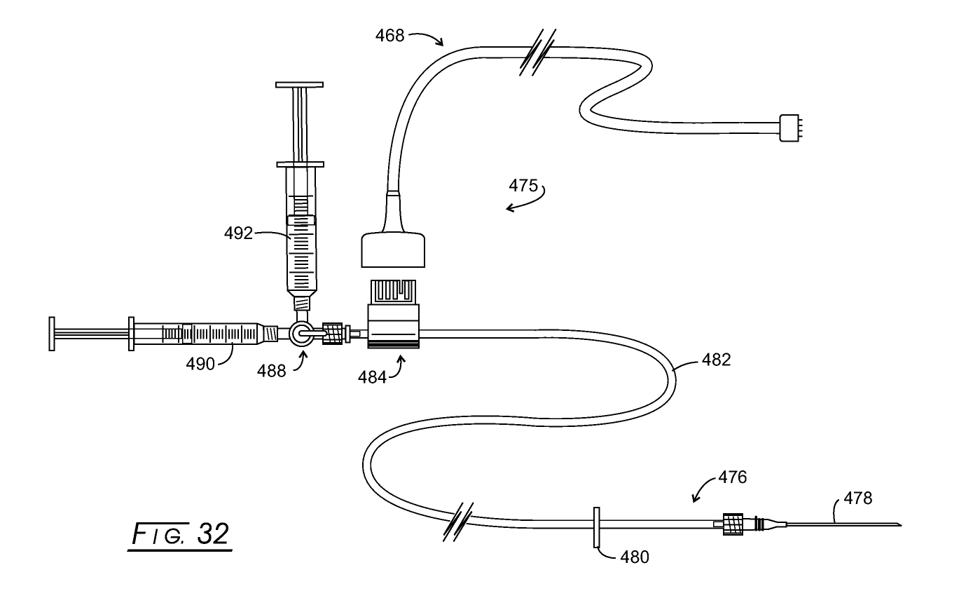

Looking to FIG. 24, a dye dilution curve 376 derived in an animal (pig) study. Curve 376 shows a recirculation effect at curve portion 378. Before computing the area represented by the curve an exponential decay represented at dashed curve portion 380 must be computed. Where a preliminary curve occurs, representing a shunt, a ratiometric analysis is made of the area under the corrected curve and the area under the shunt curve. Looking to FIG. 25, another theoretical curve is represented having a principal component 384 in association with a preliminary shunt related curve 386. The control features of the system can operate upon such curves. For example, curve 384 is reproduced in FIG. 26 in semilog fashion in conjunction with shunt curve 386. By so treating the signals, calculations can be improved in an electronic sense.

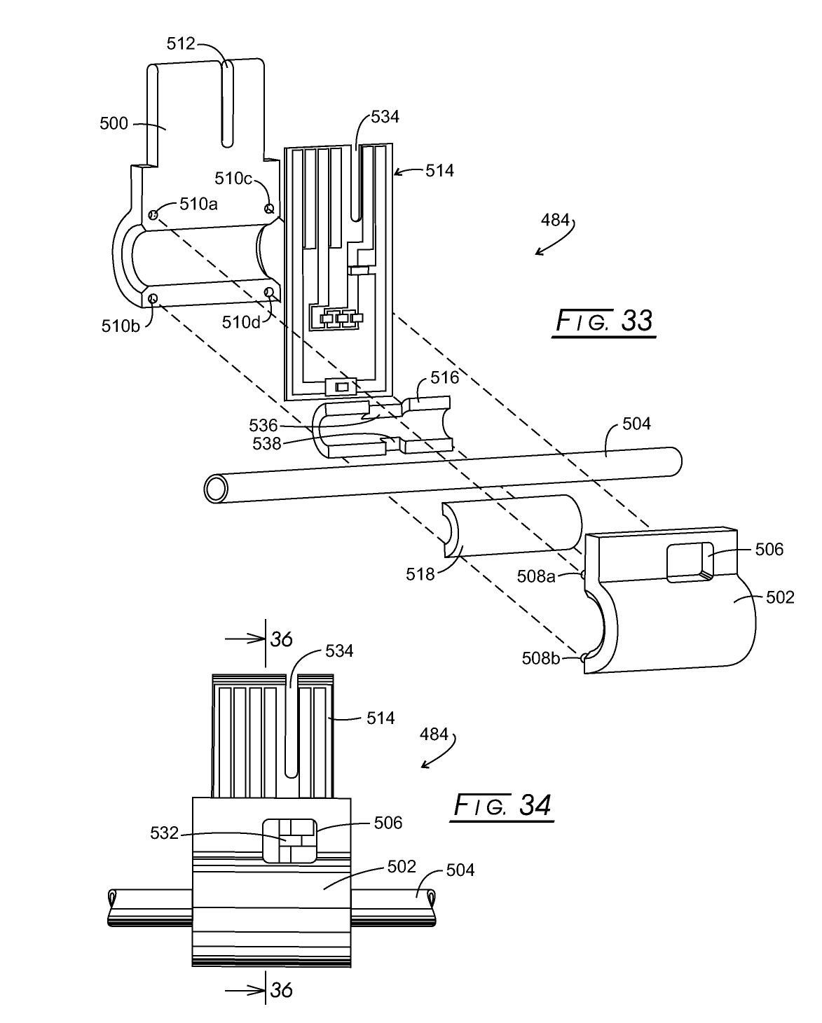

A variety of techniques are available for supporting fluorescence sensor array structures at the scaphoid fossa of the ear. In this regard, a more or less simple surgical cap has been utilized. Another approach is with a reusable headband referring to FIG. 27 such as headband set is represented generally at 390 located on the forehead of a patient 392. The front to back encircling portion of the band 390 is shown at 394, which is configured with a knob-actuated ratchet 396 for adjustment with respect to head diameter. Correspondingly, a head size height adjustable band is shown at 396 with a Velcro type fastener which may be used with a tape fastener 398, which may be used with a tape extending to a fluorescence sensor array as described at FIG. 21A and here identified at 400. Note that the device 400 is attached to the scaphoid fossa of the ear 402 and is stabilized with a Velcro type tape attached between Velcro type patches 406 and 408. Note that device 400 is similarly coupled to the right ear. Looking to FIG. 28, a head support is shown in perspective fashion in general being identified at 410. A head-encircling band is shown at 412 having a ratchet form of head size adjustment at 414. A vertical band 416 is physically attached to band 412 and incorporates a head size adjustment 418. Fluorescent sensing components are seen for right and left ears as at 422 and 424. From each of these, a respective cable 426 and 428 extends to a communication hub shown generally at 430. Hub 430 additionally connects to a controller/monitor as represented by the cable 433. Device 422 is coupled to band 416 by strap 423, while device 424 is coupled to Velcro type patch 420 by strap 421.

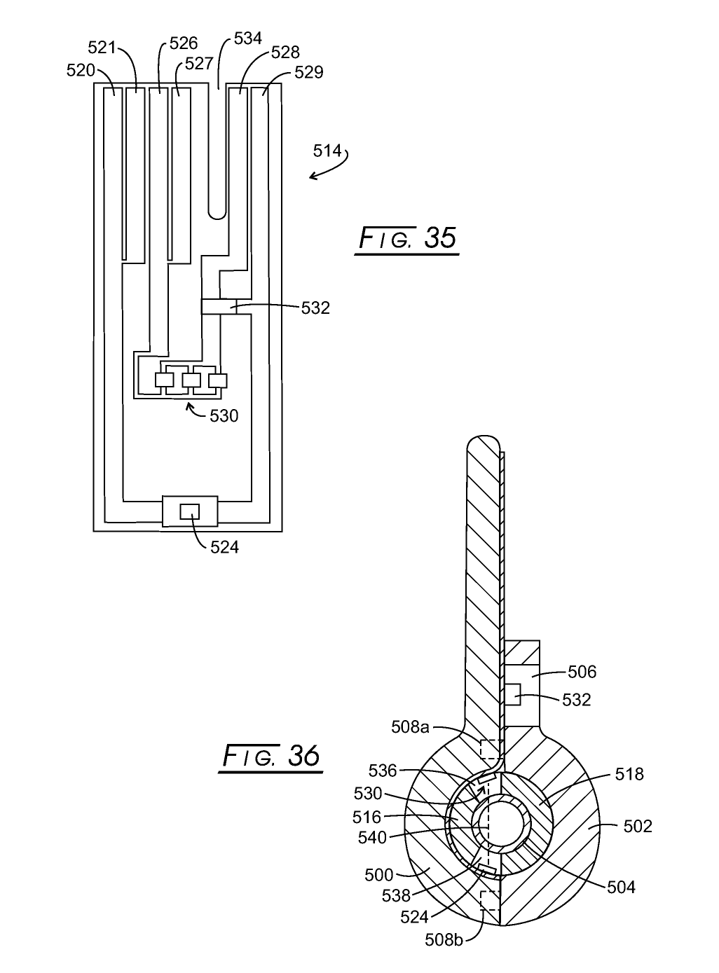

Looking to FIG. 29 the communication hub 430 is illustrated somewhat schematically with the outward cap thereof being removed. Hub 430 includes a right ear connecter 436, which receives an input as well as provides outputs to cable 426 extending from device 422. In similar fashion, a right ear connector 438 couples with cable 426. Finally, a connector for cable 432 is directed to a monitor controller as shown at 440. Circuitry is represented in general at 442.

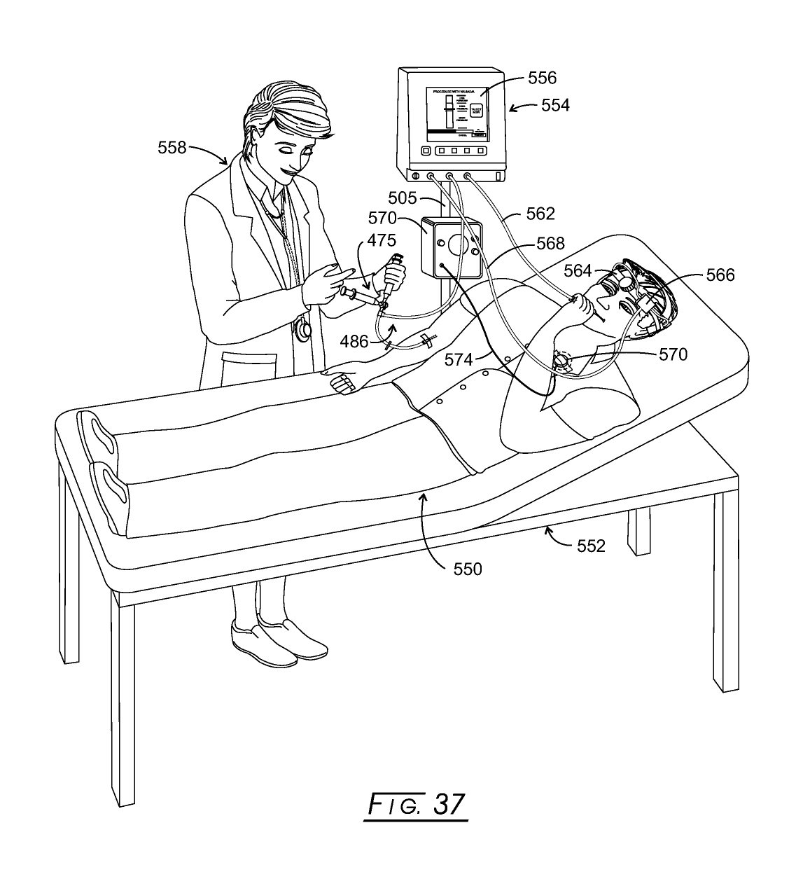

Referring to FIGS. 30 and 31, a monitor controller for use with the system is represented in general at 450. The monitor 450 may be mounted on a pole, e.g., an IV pole, includes a housing 452 which provides a display 454 which performs in conjunction with touch switches shown as an array represented generally at 456. (FIG. 30) At the bottom of housing 452 there is an input 458 for receiving the exhalation pressure occurring with a Valsalva Maneuver. Next adjacent to the input 458 is input 460 which receives an injection flow signal. Next adjacent to input 460 is 462 which is coupled with the earlier described main cable 432 extending from hub connector 440. The lasers are enabled with a key-actuated switch 464 and a flash drive recorder may be received at slot 466. Looking to the rear view at FIG. 31, the housing 452 may be pole mounted using C-type and shaped clamp 468. Electrical power input and the switching thereof is provided at switch receptacle 470.

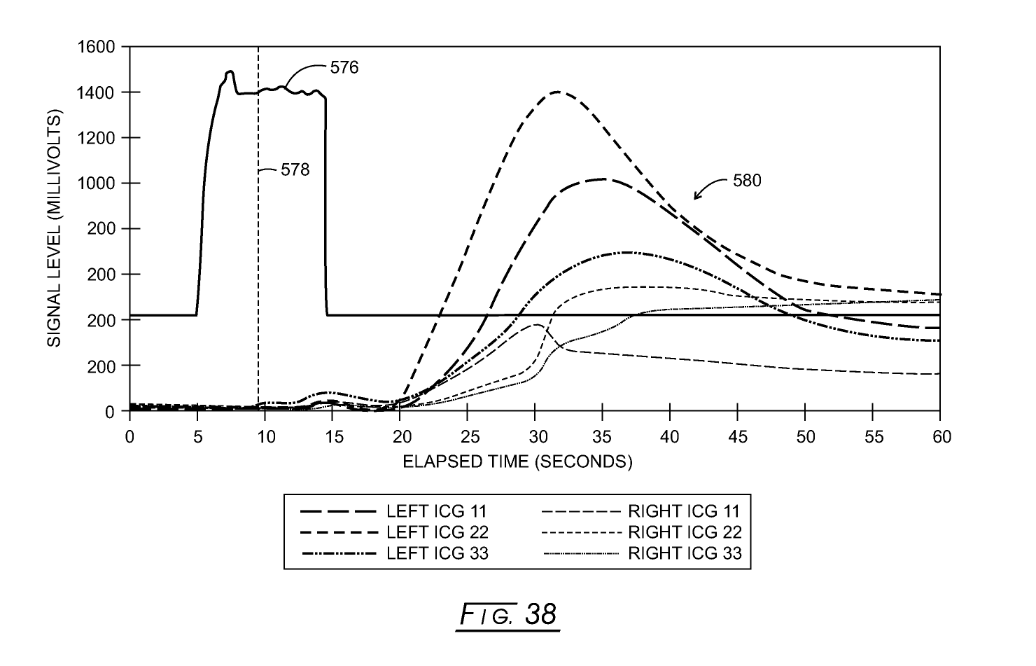

The present procedure incorporates visual and oral cueing in connection with display 450. This involves, inter alia, the placement of a vein access catheter in a peripheral vein; for example the antecubital vein in the right arm. FIG. 32 illustrates the preferred dye indicator and saline solution delivery mechanism. Looking to the figure such equipment is illustrated in general at 475. Equipment 475 includes a relatively short catheter with a 20 gauge needle as represented in general at 476, the needle being shown at 478 and a connector to main tubing being represented at 480. The principal tubing is shown at 482, a flexible elongate delivery tube extending between proximal and distal ends, with an auxiliary catheter coupled in fluid transfer relationship with the distal end defining the outlet. An indicator fluid flow detector represented generally at 484 coupled in fluid transfer relationship with the proximal end and deriving signals corresponding with the commencement and termination of fluid flow through the system. The indicator flow detector has an output signal at a cable represented in general at 486. Just upstream of flow detector 484 is a 3-way valve represented in general at 488. Connected to valve 488 is a first syringe 490, containing indocyanine green dye (ICG), which initially is injected into the principal tubing 482. Following such injection, the valve 488 is switched and saline solution from a second syringe 492 is injected to, in effect, push the ICG into the antecubical vein. Flow detector 484 detects the dye flow and provides a corresponding signal to the monitor at input 460. It is from this signal that the monitor determines the commencement of transit time.

A further embodiment of the system is a kit supplying consumable materials necessary for quantifying a circulatory anomaly comprising a one or more doses of indicator dye reagent as a shelf stable material; a saline diluent for preparing the dose of indicator dye reagent for injection into a patient; a syringe and needle apparatus for mixing the dose of indicator dye reagent and the diluent. The syringe and needle provided are suitable for injecting the indicator dye dose into the system injection port, and will typically be supplied as a first and second syringe suitable to introduce the indicator dye reagent and saline bolus into the patient. Finally, a saline solution, for instance, is provided to supply a dose of nonreactive blood compatible clearing reagent for completing the injection, and pushing the indicator dye dose into the bloodstream of the patient.

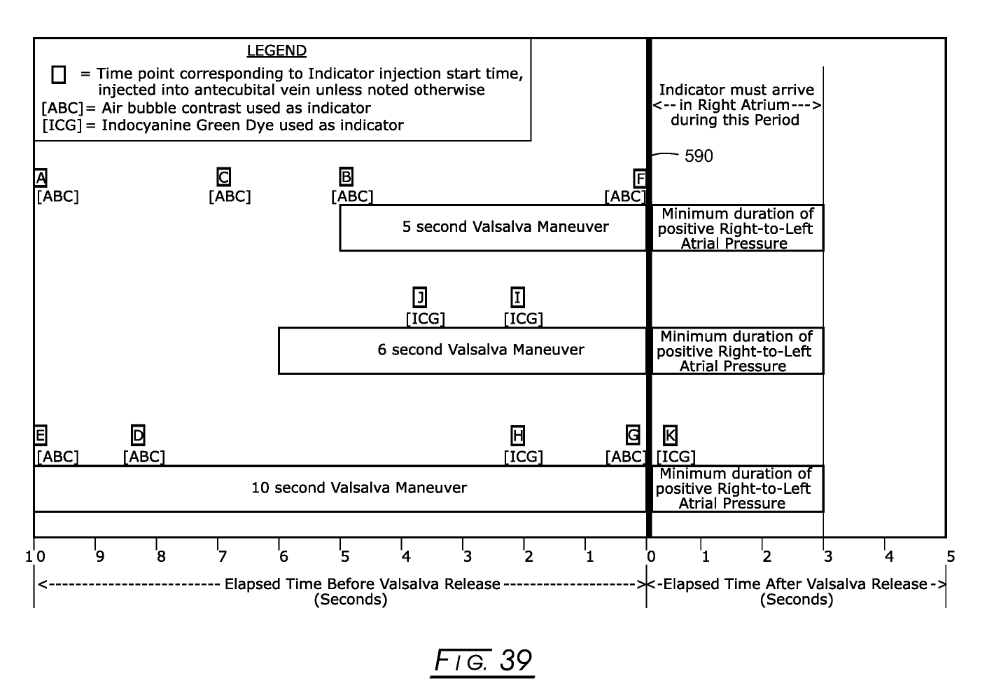

Referring to FIGS. 33 through 36, the dye flow detector 484 is revealed in enhanced detail. FIG. 33 shows two inter-connectable clamp housings 500 and 502 placed on either side of the portion of delivery tubing 504. Additionally, clamp-housing 502 is configured with 4 pins, two of which are seen at 508a and 508b. Two similar pins (not shown) are located on the opposite side of clamp housing 502. These pins are intended to be inserted within holes 510a-510b, within clamp housing 500. Note additionally that clamp housing 500 has a slot 512 formed therein, which provides connector registry. Device 484 performs a conjunction with a flexible circuit shown generally at 514. Flexible circuit 514 is retained in a wrap-around orientation by oppositely disposed support components 516 and 518.

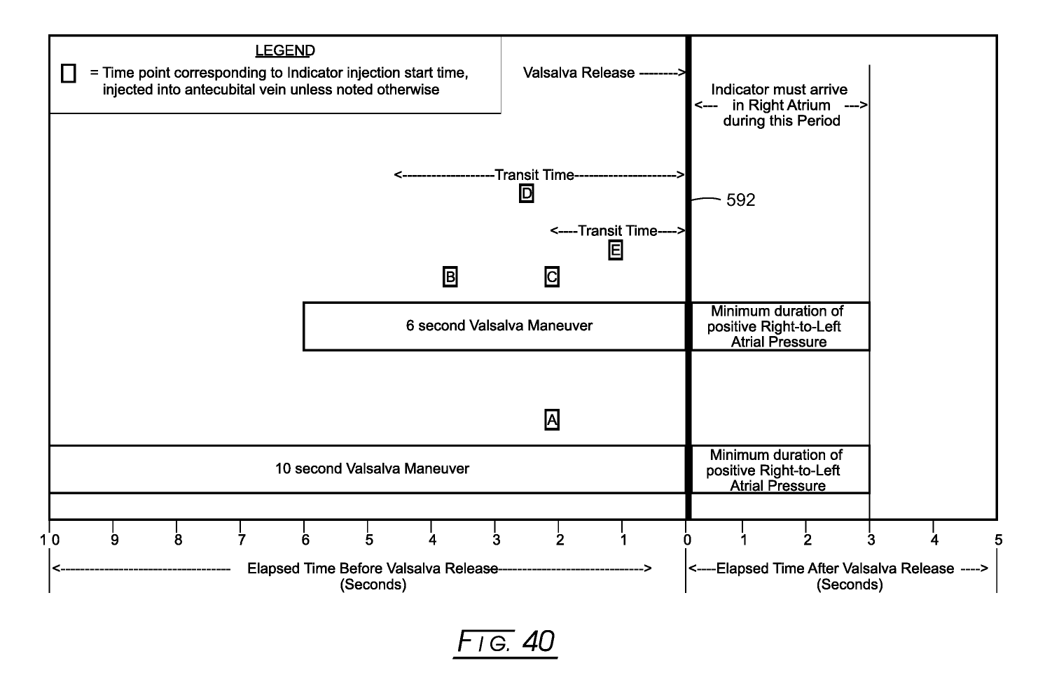

Turning to FIG. 35, the flexible circuit 514 is represented at a higher level of detail. In that figure outboard printed circuit leads 520, 521 and 529 extend to a laser 524. Leads 526, 527 and 528 extend to an array of three photodetectors shown generally at 530. A fuse 532 extends between flat leads 528 and 522. This fuse reappears in FIGS. 34 and 36. Looking to FIG. 36, laser 524 is seeing emitting laser light through the tube 504 and into the array 536. Note the alignment slot 534 in the flexible circuit 514. This slot aligns with that shown at 512 at FIG. 33. FIG. 36 shows that openings 536 and 538 in support component 516 permit the laser light distribution and reception shown at dashed line 540 in FIG. 36.

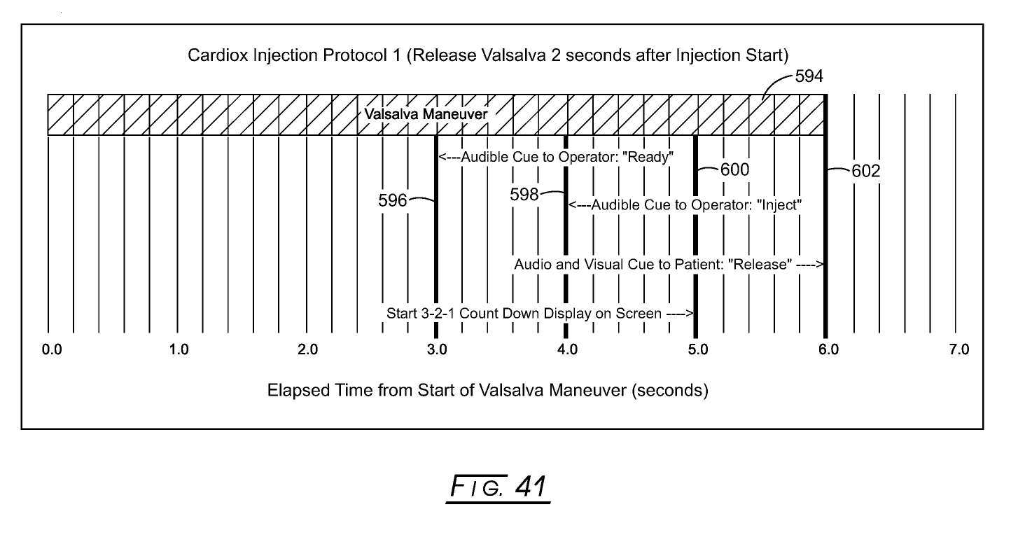

Turning to FIG. 37, a stylized representation of the present system is presented. In the figure, a patient is shown in general at 550 reclining upon an examination bench represented generally at 552. Patient 550 should either be supine or the head and trunk can be elevated about 30°, which is the arrangement depicted herein. The monitor is shown in general at 554 having a display 556 which can be observed by both the patient 550 and the practitioner represented generally at 558. Monitor 554 is mounted such that patient 550 may carry out a Valsalva Maneuver under visual cueing provided at display 556. That maneuver is carried out with a tube and mouthpiece 562, which as described earlier is connected with the monitor 554. Monitor 554 both times the Valsalva Maneuver and provides a bar chart showing when proper air exhalation pressure if present. It also indicates the Valsalva timing which may, in the instant system be about 6 seconds. Wearing a headband 564, both ears are connected with a fluorescent sensing array in the manner describing in connection with FIG. 27 and in particular the scaphoid fossa of the ear pinna. Such signals are collected at hub 566 and are directed by cable 568 to the monitor 554. Practitioner 558 is holding the injection equipment described in conjunction with FIG. 32 as is illustrated in general at 475 with a cable delivering an indicator flow providing a signal to monitor 554. The catheter arrangement 476 is shown in the instant figure having been inserted within the antecubital vein in the right arm of patient 550. Located on the left parasternal position of the chest of patent 550 is a pick-up 570 of a precordial Doppler ultrasound device shown at 572 coupled to cable 574. Device 570 preferably is positioned on the chest in combination with a coupling gel. Device 570 will provide an output at such time as saline and dye are present in the right atrium of the heart of patient 550. This confirms that the indicator has reached the right atrium and provides another indication of transit time for the indicator.

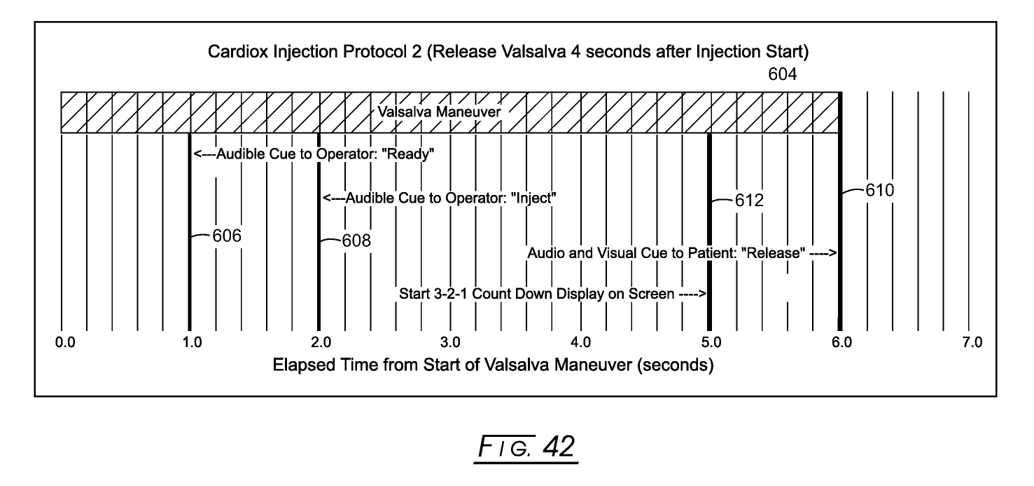

Referring to FIG. 38, the results of a human trial involving a volunteer without a right to left pulmonary shunt is displayed. The trial was configured very much like that described in FIG. 37. In this regard, the Valsalva Maneuver is represented at curve 576. During that maneuver, an injection was made at the point in time represented by the vertical dashed line 578. Fluorescent sensing arrays were mounted at each ear and dye dilution curves commenced to be formed within about five seconds. The array of six curves as identified in the tabulation is shown in general at 580. Array 580 reveals that six opportunities are present to detect indicator.

As indicated earlier herein, any indicator incorporated to discover a right to left shunt must arrive in the right atrium as the normal pressure difference between those cavities reverses. That reversal will continue for about three to five heart beats with a minimum duration of about 3 seconds. A literature study was carried out concerning the starting of the Valsalva Maneuver and the point in time when injection into a vein was made, for example, the antecubital vein. Referring to FIG. 39 results of such a literature study are presented both with respect to utilizing air bubble contrast as an indicator and indocyanine green dye as an indicator. As shown in the legend in the figure, the indicator injection start time is represented by a small square within which reference corresponding with that injection time may be listed. In the figure, “A” shows air bubble contrast being delivered before the commencement of a five second Valsalva Maneuver. This injection position reflects the works of: Droste (1999), Droste (1999a), Schwarze, (1999), Droste (2002), Devuyst (2004), Jauss (2000), Saqqur (2004), Schwarze (1997), and Schwarze (1997a). Location “B” shows the injection position at the beginning of a five second Valsalva Maneuver. The references associated with this form of injection are Zanette (1996), Schwarze (1999), Heckman (1999), Sasery (2007), Uzuner (2004), and Schwarze (1997a). Injection position “C” indicates the introduction of air bubble contrast about two seconds prior to the commencement of a five second Valsalva Maneuver. The references associated with this entry are Horner (1997), and Hamann (1998). The injection position “D” occurs about two seconds following the commencement of a ten second Valsalva Maneuver. The associated references are Karnick (1992), and Devuyst (1997). Injection position “E” occurs at the commencement of a ten second Valsalva Maneuver. The references associated with it are Zannete (1996), and Spencer (2004). Injection position “F” occurs at the end of a five second Valsalva Maneuver and the references associated therewith are Chimowitz (1991), Albert (1997), and Anzolar (1995). Injection position “G” occurs at the end of a ten second Valsalva Maneuver and the references associated therewith are Harms (2007), and Greim (2001). Injection position “H” occurs about two seconds before the end of a ten second Valsalva Maneuver and represents an injection of indocyanine green dye as an indicator. The references associated with this injection position are Banas (1971), Karttunen (1998), and Karttunen (2001). Injection timing “I” involves the injection of indocyanine green dye four seconds into a six second Valsalva Maneuver and is identified herein as Protocol Number 2. Correspondingly, injection position “J” shows an injection of indocyanine green dye, two seconds before a Valsalva Maneuver. (Protocol Number 1). Injection position “K” involves an injection of indocyanine green dye, which is injected directly into the right atrium in conjunction with a ten second Valsalva Maneuver. The reference associated with this injection is Niggemann (1987). As apparent, the Valsalva release occurs at the position represented by Vertical Line 590.

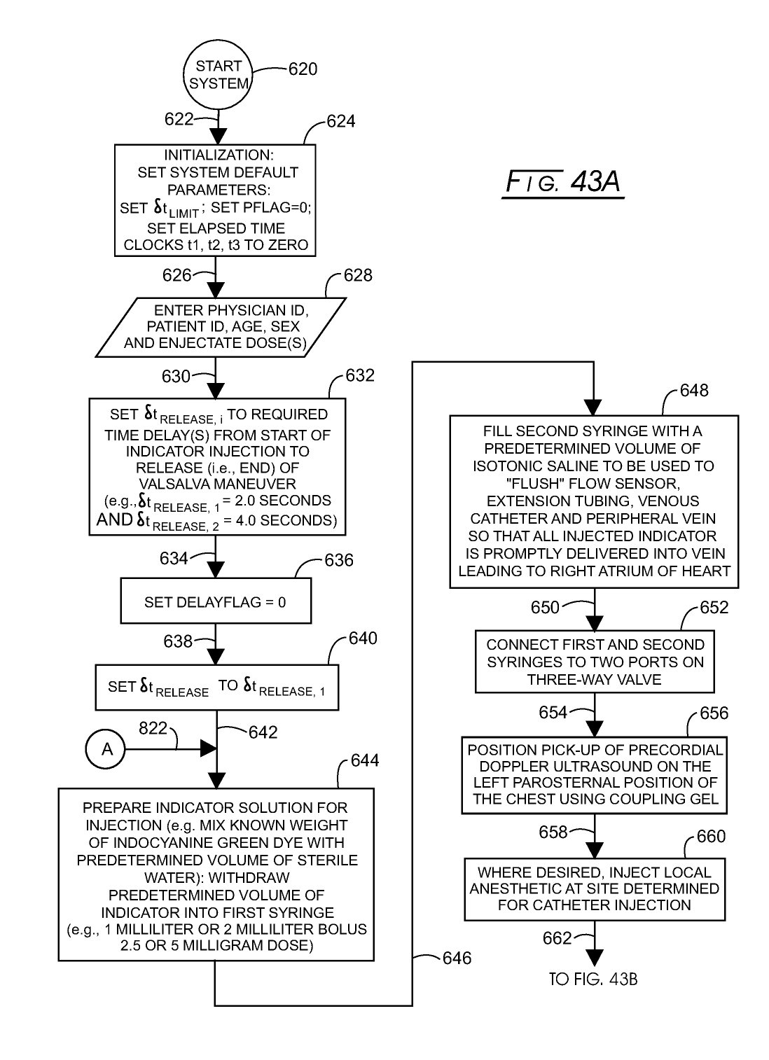

Referring to FIG. 40 another chart shows Valsalva Maneuver duration, release at Vertical Line 592 and average measured transit time. Injection position “A” is associated with a ten second Valsalva Maneuver and injection is seen to occur two seconds before Valsalva release. For references, see Banas (1971), Karttunen (1998), and Karttunen (2001). Injection “B” occurs four seconds before Valsalva release and represents an injection Protocol 1. Injection position “C” represents a Protocol 2 and occurs two seconds before Valsalva release. Transit time “D” represents the average measured transit time for indicator Bolus, injected at the antecubital vein to reach the right atrium. That transit time is reported to be 5.2 plus/minus 1.2 second, see Jauss (1994). Transit time “E” represents the average measured transit time for indicator Bolus injection at the antecubital vein to reach the right atrium. The transit time is reported to be 2.1 plus/minus 0.3 seconds. See: Teague (1991).

Referring to FIG. 41 a chart showing what is herein referred as Protocol 1 is set forth. In the figure a six-second Valsalva Maneuver is shown at Bar 594. Vertical line 596 shows that at three seconds into the Valsalva Maneuver an audible cue is given to the operator to be ready to inject indicator dye. One second later, the operator is cued audibly to inject the indicator at the antecubital vein as represented at vertical line 598. At five seconds, represented Line 600, a “3-2-1” countdown is displayed on the screen alerting both the patient and practitioner that Valsalva release will occur in one second. Such release is represented by vertical line 602 along with an audio and visual cue to the patient.

Looking to FIG. 42, a corresponding Protocol 2 is charted with respect to a six second Valsalva Maneuver as represented at Bar 604. Vertical line 606 occurs one second into the Valsalva Maneuver and it cues the operator to be ready to inject. One second later as represented at Vertical line 608 an audible cue is given to the operator to inject the indicator dye into the antecubital vein. Four seconds later, as represented at vertical line 610, an audio and visual cue is given to the patient to release the Valsalva Maneuver. One second prior to this release as represented at vertical line 612, a “3-2-1” count down is displayed at the display screen.

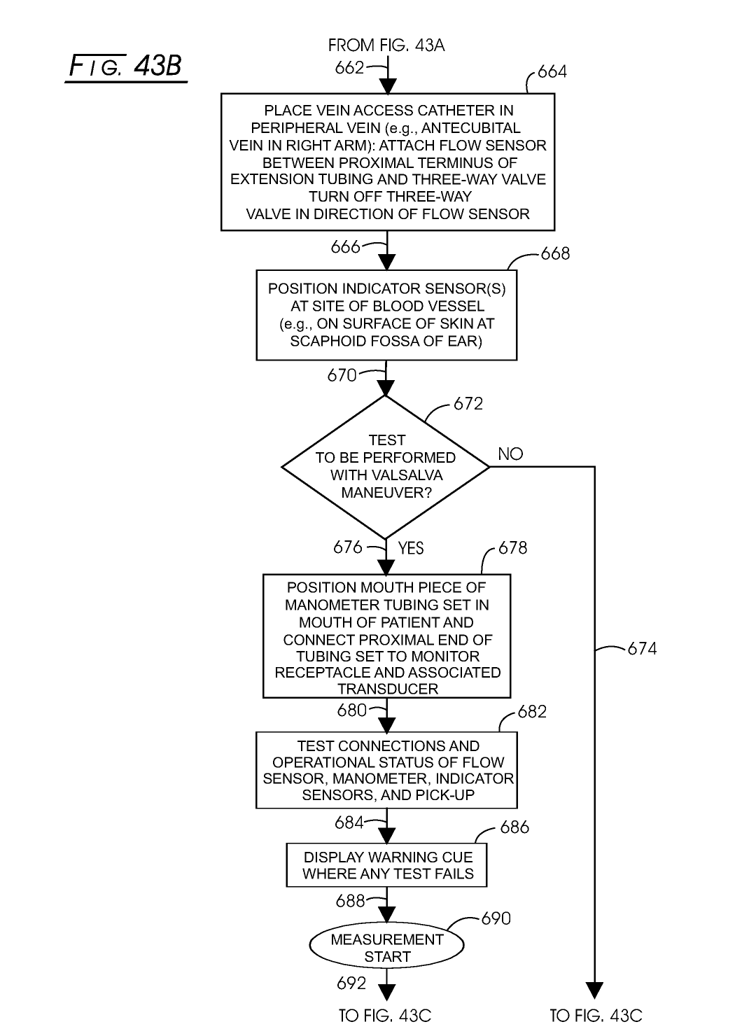

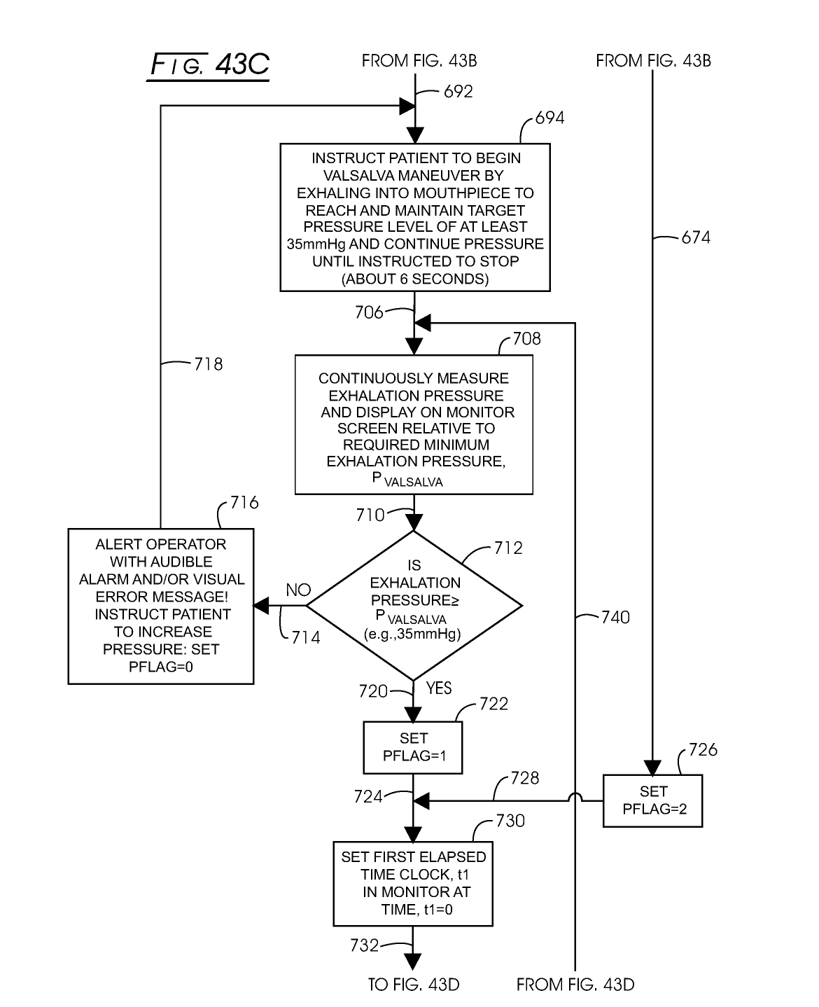

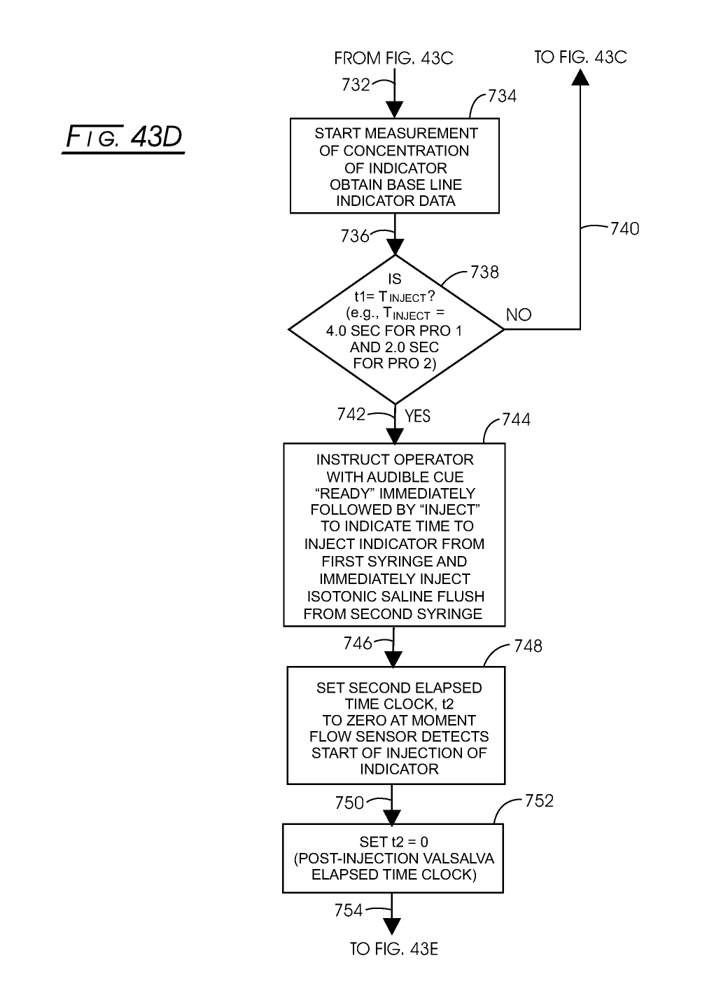

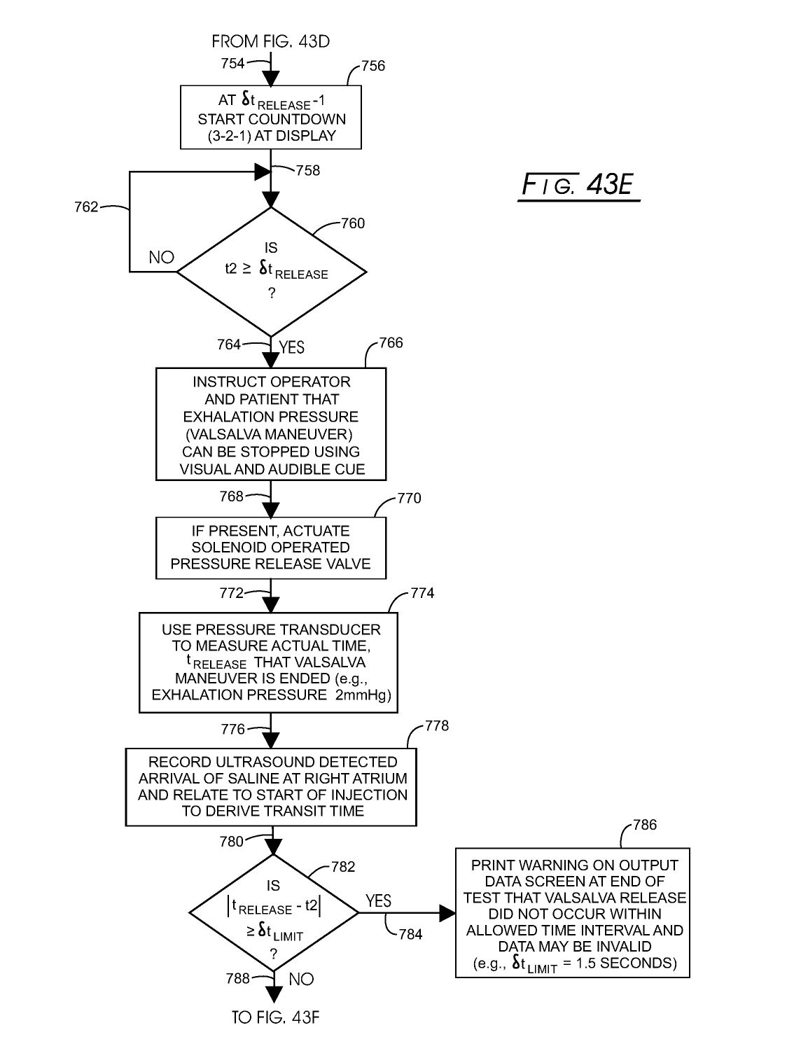

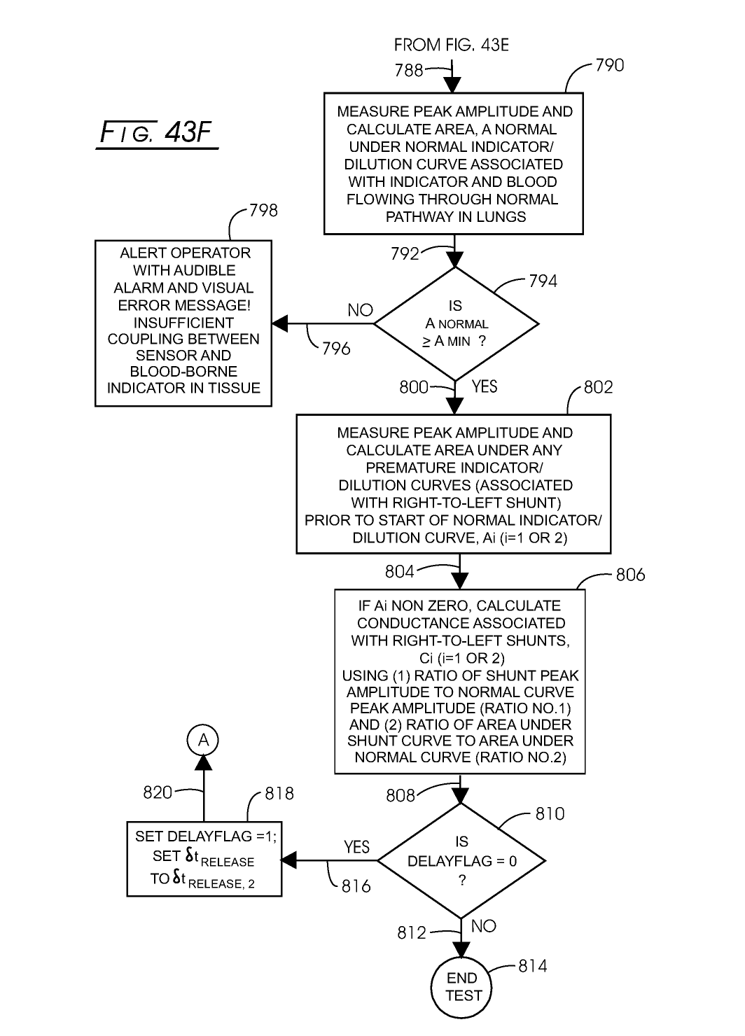

FIGS. 43A through 43F combine as labeled thereon to provide a flow chart describing the method at hand.

The method starts as represented at Symbol 620 and continues as represented at arrow 622 and block 624. At block 624, the controller carries out initialization with default parameters. The δt_{LIMIT} represents the permitted interval past the release for Valsalva that may have not been met. That being the case, any data may be invalid. PFLAG is set to zero and the elapsed time clocks t₁, t₂ and t₃ are set to zero. Next, as represented at arrow 626 and block 628, the physician identification number, the patient identification, age, sex and intended injecting doses are entered into the monitor. As represented at arrow 630 and block 632, δt_{RELEASE, i}, is set to required time delay from the start of indicator injection to Valsalva release. In this case, δt_{RELEASE, 1} is set to two seconds. As discussed in connection with FIG. 41 this is Protocol 1. The corresponding δt_{RELEASE, 2} is set to four seconds and this is the Protocol 2 described in connection with FIG. 42. Next, as represented at arrow 634 and block 636 the delay flag is set to zero and, as represented at arrow 638 and block 640, δt_{RELEASE} is set to δt_{RELEASE, 1}, or Protocol 1. The program continues as represented at arrow 642 and block 644 where the indicator solution for injection is prepared, for example mixing a known weight of indocyanine green dye with a predetermined volume of sterile water. A predetermined volume of that mixed indicator is withdrawn into a first syringe. That syringe is shown at 490 at FIG. 32. The program continues as represented at arrow 646 and block 648. Block 648 provides for filling the second syringe with a volume of isotonic saline. That isotonic saline is used to “flush” the flow sensor extension tubing catheter and the like and the peripheral vein so that all of the injected indicator is promptly delivered into the vein. As represented at arrow 650 and block 652, the two syringes are connected to ports on a three-way valve. That three-way valve has been described at 448 in FIG. 32. As represented at arrow 654 and block 656, the pickup of a precordial Doppler ultrasound device is located at the left parosternal position of the chest and a coupling gel may be used. The use of that Doppler device and pickup is represented for example at 570 and 572 in FIG. 37. From block 656 the program continues as represented at arrow 658 and block 660, the latter block describing what was found to be beneficial in that a local anesthetic may be injected at the site of intended catheter injection. The program continues as represented at arrow 652, which reappears in FIG. 43B leading to block 664.

The later block provides for placing the vein access catheter in a peripheral vein and preferably at the right arm. The flow sensor is also attached. This flow sensor has been described in connection with FIGS. 33-36 and may be utilized by the control system in conjunction with elected Valsalva start and timing to achieve an effective transit time of the indicator. The fluorescing sensing indicators then are positioned at the scaphoid fossa of the ears of the patient as represented at arrow 666 and block 668. From block 668, arrow 670 leads to the query posed at block 672 determining whether or not the test is to be performed with a Valsalva Maneuver. In the event that it is not, then the program proceeds as represented at arrow 674. In the event of an affirmative determination at block 672, then as represented at arrow 676 and block 678, the mouthpiece of the Manometer tubing set is positioned in the mouth of the patient and connected to the monitor. This is shown in FIG. 37 at tube 562 extending to an input at monitor 556. The system can be programmed also to practice the Valsalva Maneuver in conjunction with a readout at the monitor display. Next as represented at arrow 680 and block 682 the tests of connections and operational status of the flow sensor, manometer, indicator sensors and the pickup are internally made. In the event there is an error, then as represented at arrow 684 and block 686 a warning cue is made. Next, the program starts measurement as represented at arrow 688 and Symbol 690. Such measurement commences as represented at arrow 692 which reappears at FIG. 43C extending to block 694. It provides the instructions for the patient to start the Valsalva Maneuver. Generally, this is accompanied by a form of display at the monitor.

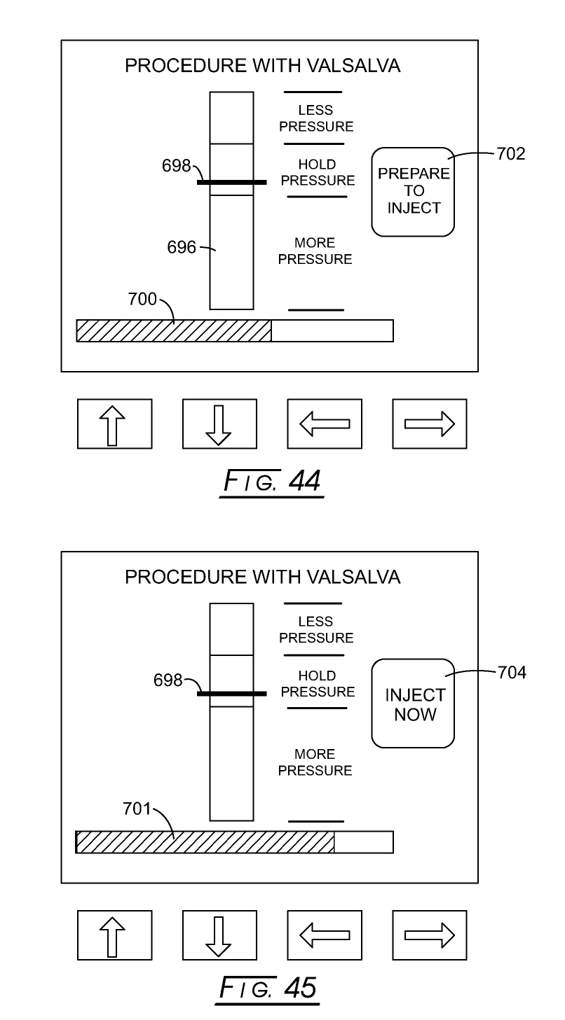

Turning momentarily to FIG. 44, a bar chart 696 is provided along with an indicator line 698 giving the patient the condition of the Valsalva Maneuver in terms of holding the correct pressure or not. A horizontal bar chart is shown at 700 showing the position in time during which the Valsalva Maneuver is carried out. For example, it may be carried out for about six seconds. The display also may forewarn the practitioner to prepare to inject as represented at 702. In FIG. 45, the proper pressure is being held and, as represented at 704, the practitioner is instructed to inject as described in connection of FIG. 41 for Protocol 1. That instruction occurs two seconds before the termination of the Valsalva Maneuver. These instructions are represented by arrow 706, block 708, arrow 710 and block 712.

Block 712 poses the query as to whether the exhalation pressure is above or equal to the targeted pressure, for example 35 mm of mercury. In the event that it is not, as represented at arrow 714, the practitioner is alerted with an audible alarm and or visual error message to instruct the patient to increase pressure to meet target. PFLAG is set to zero and the program reverts and as represented at arrow 718 to arrow 692, where the Valsalva Maneuver is retried. Where the exhalation pressure is appropriate, that is represented at arrow 720 and block 722 where PFLAG is set to 1. Then the program continues as represented at arrow 724. As represented at arrow 674 extending from the query at block 672 and leading to block 726 PFLAG is set to 2 and the program diverts as represented at arrow 728 to arrow 724. With this arrangement, the Valsalva Maneuver is bypassed to the program as shown at arrow 732 extending from block 730. Block 730 sets elapsed time clock t₁ at time t₁=0. Arrow 732 reappears in FIG. 43D extending to block 734, which looks to obtaining base line data. Then as represented at arrow 736 and block 738 the query is posed as to whether the time for instructing injection is present. In the event that it is not, then the program reverts as represented at arrow 740. Arrow 740 continues in FIG. 43C, to arrow 706. As described in connection with FIG. 41, this timing looks to the anticipated end of the Valsalva Maneuver. When the time to inject is present then as represented at arrow 742 and block 744, the practitioner is instructed, first to be ready, immediately followed by instructions to commence the injection first of the indicator, and next the isotonic saline flush. The flow sensor will detect the flow of indicator and when such flow is detected, as represented by arrow 746 and block 748, time clock t₂ is set to zero. Next, as represented at arrow 750 and block 752, the post injection Valsalva elapsed time clock t₂ is set to zero and the program continues as represented at arrow 754. Arrow 754 reappears in FIG. 43E extending to block 756. Block 756 starts the “3-2-1” countdown one second before Valsalva release. The program continues as represented at arrow 758 leading to block 760. Block 760 represents a query as to whether the post injection elapsed time clock t₂ has reached the time for Valsalva release. In the event that it has not, then the system dwells as represented by arrow 762 extending to arrow 758. Where an affirmative response is received from the query at block 760, then as represented at arrow 764 and block 766, the practitioner is instructed that the Valsalva Maneuver can be stopped, a visual and audible cue being utilized.

The monitor/controller may contain a solenoid-actuated valve, which may be activated to automatically release the Valsalva Maneuver. Accordingly, as represented arrow 768 and block 770, such valve is now opened to automatically stop the Valsalva Maneuver. The time of release also can be developed from the pressure transducer within the monitor accordingly, as represented at arrow 772 and block 774, the pressure transducer measures the actual time that the Valsalva Maneuver is ended. This may occur with an exhalation pressure dropping to 2 mm of mercury. Thus, the system provides an electromagnetically operated pneumatic valve at the monitor/controller coupled with the pneumatic tube and actuateable to a vent-to-atmosphere orientation from an open to the venting orientation and actuateable by the monitor/controller in response the provided cue.

Transit time can also be evolved as represented arrow 776 and block 778. The arrival of saline at the right atrium of the heart can be picked up and recorded to determine a transit time. From block 778, an arrow 780 is seen directed to block 782. At block 782 the query is posed as to whether the absolute value of the time of release minus t₂ is greater than of equal to the pre-designated limit time. In the event that it is, then as represented at arrow 784 and block 786, a warning is outputted at the display indicating that the Valsalva release did not occur within an allowed time interval and data may be invalid. This limiting time may, for example, be 1.5 seconds. However, such time window may be zero seconds. If the query posed at block 782 results in a negative determination, then as represented at arrow 788, the program continues to FIG. 43F. Note in that figure, that arrow 788 reappears extending to block 790. Block 790 measures the peak amplitude and calculates the area under the normal indicator dilution curve associated with indicator and blood flowing through a normal pathway in the lungs. Then, as represented at arrow 792 and block 794, a query is made as to whether the area under the normal curve is equal to or greater than a minimum designated area. Where it is not, then as represented at arrow 796 and block 798, the practitioner is alerted with an audible/visual error message that there is insufficient coupling between the sensor and blood-born indicator in tissue. Where that area is greater than the minimum area, then as represented at arrow 800 and block 802 the peak amplitude and area under any premature indicator/dilution curve, i.e. a shunt curve occurring prior to the start of the principal indicator dilution curve, are measured. Where a non-zero result curve is occurring, then as represented at arrow 804 and block 806, conductance associated with a right to left shunt is calculated. This can be done using a ratio of the shunt peak amplitude to the normal curve peak amplitude or the ratio of the area under the shunt curve to the area of a normal curve. Next, as represented at arrow 808 and block 810 an inquiry is made to whether the delay flag is now zero. Where it is not, then as represented at arrow 812 and symbol 814, the test is ended. Where the delay flag is zero, then as represented at arrow 816 the delay flag is set to one and δt_{RELEASE} is changed to δt_{RELEASE, 2}. The program then continues as represented at arrow 820 and Node A. The program is now prepared to enter Protocol 2. In this regard, Node A reappears in FIG. 43A in conjunction with arrow 822 extending to arrow 642.

The present application herewith provides reference to United States application for patent Ser. No. 12/418,866, filed Apr. 6, 2009 and entitled “Hemodynamic Detection of Circulatory Anomalies” which, in turn, makes reference to U.S. Provisional application Ser. No. 61/156,723, filed Mar. 2, 2009, and to U.S. Provisional application Ser. No. 61/080,724, filed Jul. 15, 2008, the disclosures of which are incorporated by reference. Also, all citations referred herein are expressly incorporated herein by reference. All terms not specifically defined herein are considered to be defined according to Dorland's Medical Dictionary, and if not defined therein according to Webster's New Twentieth Century Dictionary Unabridged, Second Edition.

Since certain changes may be made in the above-described system, apparatus and method without departing from the scope of the invention herein involved, it is intended that all matter contained in the description thereof or shown in the accompanying drawings shall be interpreted as illustrative and not in a limiting sense. The disclosed invention advances the state of the art and its many advantages include those described and claimed.

We claim:

1. A method for detecting the presence of a right-to-left pulmonary shunt in a patient, comprising the steps:
providing an indicator delivery system having an outlet located in a vein of the patient in blood flow communication with the right side of the heart and actuateable to define an anticipated transit time substantially from the commencement of delivery of indicator toward the vein and the arrival of such indicator at a pulmonary location such as the right side of the heart;
providing a sensor positionable to sense the presence of at least a portion of the indicator at arterial vasculature of one or symmetrically paired distal locations of the patient and having one or more outputs corresponding with the instantaneous concentration of indicator at such vasculature;
providing a monitor/controller having a display and responsive to said actuation to commence timing the anticipated transit time, responsive to a sensor output to display one or more indicator dilution curves to determine whether a shunt is present.

2. The method of claim 1 wherein the paired distal location is one or more ears, the hand, the neck, the leg, and the arm.

3. The method of claim 1 further comprising the steps:
providing a manometer with said monitor/controller having an air pressure responsive input and a corresponding pressure output signal;
providing a pneumatic tube with a mouthpiece engageable with the mouth for receiving the exhalation of a Valsalva Maneuver;
determining an anticipated transit time;
determining the interval of said Valsalva Maneuver; and
configuring the monitor/controller to display the start and cue the release of the determined Valsalva Maneuver and to cue the time of actuation of the indicator delivery system with respect to such start and release.

4. The method of claim 3 further comprising the steps:
providing an electromagnetically operated pneumatic valve at the monitor/controller coupled with the pneumatic tube and actuateable to a vent-to-atmosphere orientation from an open to the venting orientation and actuateable by the monitor/controller in response the cue.

5. The system of claim 3 in which the monitor/controller is responsive to publish the normal indicator/dilution curve and any premature indicator/dilution curves at its display.

6. The system of claim 1 in which:
the indicator delivery system is actuateable to inject a fluorescing biocompatible dye excitable by tissue penetrating excitation radiation to derive fluorescence emission corresponding with the indicator concentration; and
the sensor comprises a photodiode energizable to generate light at the excitation radiation wavelength and a photodetector which is filtered for response substantially only to the fluorescence emission.

7. The system of claim 1 in which:
the monitor/controller is responsive to compare the calculated area Aₙₒᵣₘₐₗ with a minimum value area Aₘᵢₙ, and is responsive to generate an audible alarm, error message and prompt when Aₙₒᵣₘₐₗ is less than Aₘᵢₙ.

8. The system of claim 1 in which:
the indicator delivery system includes a flow sensor responsive to derive signals corresponding with the commencement and termination of fluid flow through the system; and
the monitor/controller is responsive to such commencement and termination signals to derive an audible alarm to the operator when the time interval of indicator injection is excessive.

9. The system of claim 6 in which the indicator delivery system injected fluorescing biocompatible dye is indocyanine green dye.

10. The system of claim 6 in which the sensor further comprises a sensor array with transmission mode sensing in which:
the sensor array comprises two or more paired excitation laser diodes and filtered photodetector and energizable in a sequence of such pairs; and
the monitor/controller is responsive to elect that pair exhibiting a concentrator output of highest intensity.

11. The system of claim 10 in which the sensor array further comprises the laser diodes arranged with an aspheric collimating lens, a collimator plate and an interference filter in the transmission path to the photodetector.

12. The system of claim 6 in which:
the sensor array excitation laser diodes are energizable to emit light at a wavelength of 785 nanometers.

13. The system of claim 2 in which the sensor positionable at paired distal locations further comprises two fluorescence sensing array fixtures with biased sensing array arms, removably attached to a headband.

14. The system of claim 13 wherein the paired distal location is at the scaphoid fossa of ears of the patient.

15. The system of claim 1 in which:
the indicator delivery assembly comprises a flexible elongate delivery tube extending between proximal and distal ends, an auxiliary catheter coupled in fluid transfer relationship with the distal end defining the outlet, a indicator fluid flow detector coupled in fluid transfer relationship with the proximal end and deriving signals corresponding with the commencement and termination of fluid flow through the system, a three-way valve connected upstream to the fluid flow sensor, a first indicator containing syringe coupled in indicator flow relationship with the valve and actuateable to cause indicator to flow through the valve, and a second isotonic saline fluid containing syringe coupled in fluid flow relationship with the valve and actuateable to cause isotonic saline to flow through the valve; and
the monitor/controller is responsive to cue the operator first to actuate the first syringe and immediately thereafter to actuate the second syringe, and is responsive to monitor the corresponding fluid flow sensor signals.

16. A sensing array apparatus comprising
(a) a plurality of laser diode emitter and photodetector pairs for monitoring the fluorescence of a fluorescing circulatory tracking reagent;
b) said laser diode emitters providing a excitation light source emitting a first wavelength for excitation of an indicator within the tissue of a patient body, the emitters transmitting the excitation light through a collimator lens having a collimating channel aligned with an optical path an interference filter, said collimating channel and interference filter located intermediate to said laser diode emitter and photodetector; and
(c) said detectors for measuring the intensity of the fluorescent light emitted by the tracking reagent at a second wavelength from an excited indicator within the blood stream; and,
(d) a clamping array support system of a plurality of array support arms, biased in a clamping arrangement;
wherein the clamping array support system can placed in a clamping arrangement on the exterior of the patient body, whereupon activation of one or more of the laser diode emitters said laser diode emitters transmit excitation light through tissue of the patient, thereby exciting indicator present, said photodetectors measuring the intensity of light emitted by excited indicator.

17. The sensing array apparatus of claim 16 wherein the plurality of laser diode emitter and photodetector pairs are three laser diode emitter and photodetector pairs.

18. The sensing array apparatus of claim 16 wherein two sensing array apparatuses are used at symmetrical locations on the human body.

19. The sensing array apparatus of claim 16 further comprising an interlock component comprised of a light emitter and photodetector pair disposed on the array support arms such that positioning of the clamping array support system in the proper clamping arrangement brings the light emitter and photodetector pair in close apposition, providing an optical interlock having a signal utilized by the control circuitry associated with the sensing array apparatus.

20. A pulmonary anomaly detection system wherein a biocompatible indicator is controllably introduced into a peripheral vein of a patient having one or more ears, each with a helix partially peripherally surmounting a scaphoid fossa, such indicator being excitable by energy at a first wavelength to emit fluorescent energy of a second, higher wavelength, such system having a transmission mode sensing device, comprising:
a first branch with an excitation assembly operationally engageable with one surface of an ear scaphoid fossa and having at least one laser energizable to emit photon energy at said first wavelength along one or more optical paths and a one or more corresponding collimating lenses, each disposed within an optical path of a laser directing collimated photon energy at said first wavelength through said one surface;
a second branch with a sensor assembly corresponding with said excitation assembly operationally engageable with the ear scaphoid fossa at another surface opposite said one surface and having a photodetector aligned with each optical path excitable by impinging photons to derive an intensity signal, an interference filter located between said other surface opposite said one surface and a photodetector and exhibiting a bandpass corresponding with said fluorescent energy at the second higher wavelength; and
said first and second branches being mechanically biased toward each other.

21. The system of claim 20 wherein said sensor assembly further comprises:
a collimator having a collimating channel aligned with an optical path and located intermediate the other surface of the ear scaphoid fossa and an interference filter.

22. The system of claim 20 in which:
each said first and second branch respective excitation assembly and sensor assembly comprises respectively, an array of two or more lasers and corresponding two or more photodetectors; and
said first and second branches are pivotally joined together.

23. The system of claim 22 in which:
said first and second branches are cooperatively configured to have an optical interlock formed with a light emitting diode in one branch with a light output along an interlock optical path and a photodetector aligned with the interlink optical path and located in the opposite branch.

24. The system of claim 20 in which:
each said excitation assembly and sensor assembly is mounted with a mutually inwardly depending protrusion configured to extend over an ear helix to inwardly engage a surface of the adjacent scaphoid fossa.
